(19) United States
(12) Patent Application Publication
(10) Pub. No.: US 2007/0054326 A1
(43) Pub. Date: Mar. 8, 2007

(54) Antibody-based protein array system

(76) Inventor: Ruo-Pan Huang, Norcross, GA (US)

Correspondence Address: THOMAS, KAYDEN, HORSTEMEYER & RISLEY, LLP, 100 GALLERIA PARKWAY, NW, STE 1750, ATLANTA, GA, 30339-5948, US

(21) Appl. No.: 11/491,802

(22) Filed: Jul. 24, 2006

Related U.S. Application Data
(63) Continuation of application No. 10/275,696, filed on Nov. 7, 2002, now abandoned.

(51) Int. Cl. G01N 33/53; C12M 3/00

(52) U.S. Cl. 435/7.5; 435/287.2; 977/902

(57) ABSTRACT

The invention of novel protein microarrays and protein microarray-based techniques to determine the presence and amounts of proteins of interest are described. These microarrays and methods of use can be used for the simultaneous detection of a multiplicity of antigens or antibodies in a high throughput assay based upon the differential affinity of molecules for one another. The microarrays can be formed by immobilizing capture proteins in an array on a membrane. Analytes of interest can be bound by the capture proteins and can be detected either by the position to which they are immobilized or by the identity of detecting proteins or agents which bind to the analytes of interest. The interactions that can be detected using the present invention can also be used to characterize proteins of unknown identity or character.

### BACKGROUND OF THE INVENTION

1. Field of the Invention

This invention relates to the analysis of polypeptides and proteins. More specifically, the invention relates to a novel method for the detection and identification of proteins by use of an antibody-based protein array assay system, which has the advantages of specificity associated with enzyme-linked immunosorbent assays (ELISA), of sensitivity associated with enhanced chemiluminescence (ECL) and of high-throughput associated with microarrays without requiring sophisticated equipment.

2. Background of the Art

The progress of the scientific establishment in sequencing the human genome and that of other organisms has been remarkable. Soon, the genomes of many organisms will be mapped and sequenced in their entirety. All of the component parts of each cell, as defined by the information encoded in the DNA, will be known. Still, the ability to understand the coordination of gene expression and the actual function of the cell will remain a mystery. For, even though each cell's DNA is an archive of the living organism's genome from which it is taken, it is the proteins that are encoded by that DNA that do almost all of the work of the cell. Furthermore, experimental evidence clearly shows a significant disparity between the relative abundance of genes, the relative expression levels of mRNA and the levels of corresponding proteins. For these reasons, it is clear that a complete understanding of both normal and disease states in organisms will require much information beyond what can be supplied from genomic analysis; a complete understanding of cells will require a precise and accurate evaluation of protein expression.

The field of proteomics with its underlying goal of assembling a complete library of all proteins, has a vital need for tools which will allow researchers to detect and analyze large numbers of proteins simultaneously. Tools for high throughput analysis of proteins in a manner like that enabled by DNA microarrays for gene analysis are simply not available. The two state of the art methods in proteomics, two-dimensional gel electrophoresis coupled with mass spectrometry and surface-enhanced laser desorption and ionization (SELDI), both suffer from limited throughput, great complexity and high cost.

Thus, the invention described and disclosed herein, a novel method of analyzing proteins and other biomolecules, provides a much more capable method to analyze and evaluate protein expression than current analytical and diagnostic methods and does so with less complexity and at lower cost. This invention would, therefore, be of great use in basic research, in medical diagnosis and in the biotechnology industry.

### SUMMARY OF INVENTION

It is an object of this invention to provide a method for the simultaneous detection of a multiplicity of antigens or antibodies in a high throughput assay based upon the differential affinity of molecules for one another.

In a particular embodiment, the present invention provides a method for detecting a specified protein comprising: immobilizing capture proteins in an array on a membrane; passing a solution containing proteins over the array of capture proteins, thereby capturing certain proteins; adding a detection protein which binds to a specified protein, or portion thereof; and detecting bound detection protein, the detection of bound detection protein indicating the presence of the specified protein, or portion thereof.

In a particular embodiment, the present invention provides a method for detecting a specified protein comprising: immobilizing capture proteins in an array on a membrane; passing a solution containing detectable proteins over the array of capture proteins, thereby capturing certain proteins; and detecting bound detectable proteins.

In a particular embodiment, the present invention provides a method for characterizing proteins by determination of interactions between the protein to be characterized and other proteins with defined properties. For example, use of an antibody specific for a given epitope can be used to determine that a protein of interest comprises the given epitope if the antibody binds to the protein of interest. In a further embodiment, an antigen or antibody of interest is immobilized to a specific position on a microarray by specific binding interactions to a membrane-bound capture protein wherein the character of the capture protein provides information about the character of the protein of interest. In another embodiment, the binding of an antigen or antibody of known character to an antigen or antibody of interest immobilized on a membrane microarray can be used to provide information about the membrane-bound antigen or antibody of interest.

The summary of the invention described above is not limiting and other features and advantages of the invention will be apparent from the following detailed description of the preferred embodiments, as well as from the claims.

### BRIEF DESCRIPTION OF THE FIGURES

FIG. 1. Schematic representation of ELISA and ECL format to detect specific antigens in protein arrays. Antigens consisting of purified IgGs are immobilized on the PDVF membranes. Membranes are blocked with BSA. Specific antigens are detected using corresponding specific antibodies coupled with ECL.

FIG. 2. Raw image data for detection of individual antigen. 100 μl of individual IgG (50 μg/ml), TBS (negative control), BSA (negative control) and HRP-conjugated anti-Bovine IgG (as positive control) were immobilized onto PDVF membrane as indicated in the figure. The stripes were then incubated with different HRP-conjugated anti-IgGs (5 ng/ml) or without anti-IgG. The signals were then visualized by ECL. Abbreviations: Bov, bovine; Ck, chicken; Gt, goat; GP, guinea pig; Hu, human; Ms, mouse; Rb, rabbit; Shp, sheep.

FIG. 3. Sensitivity of detection. Different IgGs, negative control (TBS and BSA) and positive control (HRP-conjugated anti-Bovine IgG) were spotted onto PDVF membrane. The stripes were then incubated with different concentrations of HRP-conjugated anti-Bovine IgG and subjected to ECL. Images were obtained by short exposure (10 seconds) and long exposure (5 minutes).

FIG. 4. Detection of an entire 96-well array. 100 μl of different Igs (50 μg/ml) were immobilized onto PDVF membranes. Each row contains one individual antigen. Individual membrane was then incubated with the indicated HRP-conjugated antibodies (5 ng/ml).

FIG. 5. Schematic representation of Sandwich ELISA and ECL format to detect specific cytokines or growth factors in protein arrays. Specific capture antibodies are immobilized on PDVF membranes. Specific antigens are captured by the correspondingly immobilized antibodies. Biotin-conjugated detection antibodies then specifically bind to specific antigens. All detection antibody-binding events are indirectly detected using streptavidin-HRP and ECL.

FIG. 6. Specific detection of MCP-1 and IL-2 by sandwich format ELISA. The stripes immobilized with different concentrations of captured anti-MCP-1 antibody or anti-IL-2 antibody and other control reagents were incubated with MCP-1 (5 μg/ml), or IL-2 (5 μg/ml), or EGF (5 μg/ml), or mock. After washing, biotinylated anti-MCP (0.5 μg/ml), or biotinylated anti-IL-2 (0.5 μg/ml), or a biotinylated control antibody was then incubated with the stripes. The unbound biotinylated antibodies were then washed out and the stripes were incubated with HRP-conjugated streptavidin. The signals were then revealed with ECL kit.

FIG. 7. Sensitivity of detection of MCP-1. The stripes spotted with capture anti-MCP-1 antibody were incubated with serial dilutions of MCP-1. The signals were then detected as described in Materials and Methods.

FIG. 8. Simultaneous detection of multiple cytokines. Different capture antibodies or controls were loaded onto PDVF membranes. Membranes were then incubated with different combinations of cytokines and their corresponding biotin-conjugated antibodies.

FIG. 9. Detection of MCP-1 from conditioned medium. The membranes immobilized with different capture antibodies against different cytokines were incubated with 0.10 fold diluted conditioned media from U251cx43 (a cx43-transfected human glioblastoma cell line) and U251N23 (a control-transfected human glioblastoma cell line). After washing, the membranes were incubated with a combination of biotin-conjugated antibodies as indicated in the Figure. Signals were detected by ECL system.

FIG. 10. Selection of membranes for protein arrays. Different membranes were spotted with IgGs and other controls as indicated. The membranes were then incubated with HRP-conjugated anti-bovine IgG after being blocked with 5% BSA. The signals were visualized with ECL.

FIG. 11. Specificity and sensitivity of detection of HRP-conjugated anti-species-specific IgGs. (A) MSI magnagraph membranes immobilized with different species-specific IgGs were incubated with individual HRP-conjugated anti-IgGs against specific species or controls as indicated in the figure. HRP-conjugated anti-bovine IgG was spotted onto membranes enable orientation of blots. (B) Different concentrations of HRP-conjugated anti-bovine IgG as indicated beside each microarray row were used to test the detection sensitivity.

FIG. 12. High-density protein arrays to detect HRP-conjugated antibody. (A) Array layout. Membranes were spotted with different IgGs or controls as indicated in the array layout. There are 504 spots in one array. (B) Membranes were incubated individually or collectively with HRP-conjugated antibodies as indicated in the figure.

FIG. 13. Detection of cytokines in array format with high specificity and sensitivity. (A) Hybond ECL membranes immobilized with different capture antibodies against different cytokines were incubated subsequently with single cytokine or all six cytokines or controls as indicated in figure, with corresponding biotin-conjugated anti-cytokines or controls and with HRP-conjugated streptavidin. The signals were detected by ECL. (B) High sensitivity of detection is exemplified by IL-2. Membranes spotted with cytokines were incubated with different concentrations of cytokines. The signals were then detected with biotin-conjugated anti-IL-2 and HRP-conjugated streptavidin.

FIG. 14. Detection of cytokines in high-density protein array format. (A) Different cytokines or controls were spotted on Hybond ECL membranes in high-density array format as indicated in figure. (B) Detection of cytokines with high-density array format was demonstrated by incubation of the array membranes with individual or collective cytokines.

FIG. 15. Specific and sensitive detection of antibodies in array format. (A) MSI magnagraph membranes loaded with different IgGs as indicated were incubated with single donkey anti-species-specific IgGs or all eight antibodies. Membranes were then incubated with HRP-conjugated anti-donkey IgG. (B) Different concentrations of donkey anti-mouse IgG were used to test the detection limit in array format.

FIG. 16. High-density protein arrays to simultaneously detect multiple antibodies. Different IgGs or controls were loaded onto Hybond ECL membranes in an array format indicated in FIG. 16A. Simultaneous detection of multiple antibodies was demonstrated as indicated in FIG. 16B.

FIG. 17. (A) Detection of cytokines in array format with high specificity. Hybond ECL membranes immobilized with different captured antibodies against different cytokines were incubated subsequently with individual cytokine or control as indicated in figure, with corresponding biotin-conjugated anti-cytokine or control and with HRP-conjugated streptavidin. The signals were visualized by ECL. (B) Detection of cytokines in array format with high sensitivity. Cytokine array membranes were incubated with different concentrations of MCP-1 and IL-2. The intensities of signals were detected by densitometry and plotted against the concentrations of MCP-1 or IL-2.

FIG. 18. Simultaneous detection of multiple cytokines in an array format. The combinations of multiple cytokines as indicated in FIG. 18 were incubated with cytokine array membranes. The membranes were then incubated with combination of multiple biotin-conjugated anti-cytokine antibodies. Signals were then visualized by ECL kit.

FIG. 19. Detection of multiple cytokine expression from conditioned media. The cytokine array membranes were incubated with 50 fold diluted conditioned media from human glioblastoma cells U251 treated with or without TNFα. The membranes were then incubated with a cocktail of biotin-labeled antibodies against all twenty-four cytokines and signals were detected by ECL.

FIG. 20. Detection of cytokine expression from patient's sera. 10 fold-diluted patient's sera were incubated with cytokine array membranes and bound species were detected using ECL.

FIG. 21. Conditioned medium arrays. (A) Conditioned medium array design. (B) 0.5 μl of conditioned media from different sources were spotted onto the anti-MCP-1-coated membrane. The membrane was then incubated with biotin-conjugated anti-MCP-1 and HRP-conjugated streptavidin. The signals were then detected by ECL. C. The intensities of signals derived from standard MCP-1 were determined by densitometry and plotted against concentrations of MCP-1.

FIG. 22. The human cytokine array systems to study the molecular mechanism of tumor suppression. A). A template of human cytokine arrays. B. Human cytokine arrays from Cx43-transfected cells and control-transfected cells. C). The immunoprecipitated complex separated by SDS PAGE and the levels of MCP-1 protein were detected by Western Blot using antibody against MCP-1. D). ³H thymidine assay indicates that MCP-1 is involved in the Cx43-mediated cell proliferation.

FIG. 23. IL-8 was specifically up-regulated in human breast cancer cells harboring mutant type p53. A). Human cytokine arrays were used to profile cytokine expressions in a variety of human breast cancer cells. Conditioned media containing equal amounts of protein were subjected to protein arrays as described in Materials and Methods. B). The intensities of resulting signals were determined by densitometry. The relative expressions of MCP-1 were shown. C). Concentrations of IL-8 from conditioned media were determined by ELISA and the relative expression levels were shown. D). Cell proliferation rates were performed in the presence of antibody against IL-8 by using ³H thymidine incorporation assays as described in experimental procedures. Antibody against IL-8 specifically inhibited cell proliferation in cells expressing high levels of IL-8 (BT-20), but not in the cells expressing low amount of IL-8 (MCF-10A).

FIG. 24. Supplementation of vitamin E led to down-regulation of MCP-1 expression. A). Representative of cytokine arrays from vitamin E-supplemented patient sera. B). The relative expression levels of MCP-1 were determined by densitometry and plotted from 11 individuals involved in this study. C). The different MCP-1 levels were analyzed statistically by student T-test.

FIG. 25. Human cytokine array system adapted to protein chips platform. A). A map for the location of captured antibodies spotted onto protein chips. B). Protein chips printed with 43 cytokine captured antibodies were incubated with samples from different sources: conditioned medium (human breast cancer cells BT549), serum, cell lysate (human MDA-MB-231), tissue lysate (human endometriosis tissue), urine (a normal individual), urine (a normal individual plus purified recombinant cytokines as indicated in the Fig.) and a control (PBS).

FIG. 26. Schematic representation of biotin-labeled and antibody-based protein arrays. Samples containing proteins are labeled with Biotin. The proteins of interest are captured by antibodies arrayed on protein chips. The signals are detected by Cy3- or cy5-streptavidin and laser scanner.

FIG. 27. A) Location of captured antibody spot in human cytokine chips; B) Specific detection of individual cytokine labeled by biotin in human cytokine chips. Recombinant cytokine was labeled with biotin. The human cytokine chips were incubated with individual biotin-conjugated cytokines or with a control as indicated in the figure. The detectable signals were generated by incubation with cy3-streptavidin and the images were visualized by laser scanner. C). Simultaneous detection of multiple cytokines in human cytokine chips with high specificity. Human cytokine chips were incubated with different combinations of multiple cytokines labeled with biotin as shown in FIG. 2C. The chips were then incubated with cy3-conjugated streptavidin. Signals were visualized by laser scanner.

FIG. 28. Detection of cytokines in array format with high sensitivity. A). Human cytokine array chips were incubated with different concentrations of IL-6, IL-8, MCP-3 and TNFα labeled with biotin. The intensities of signals were detected by laser scanner and plotted against the concentrations of IL-6, IL-8, MCP-3 and TNFα. B). A representative result (for IL-8) is shown.

FIG. 29. Detection of multiple cytokine expression from real biological samples using biotin-labeled and antibody-based array system. The human cytokine chips were incubated with 50 μl of biotin-labeled conditioned media from human breast cancer cells (MDA-MB-231), patient's plasma and BT549 cell lysate. The chips were then incubated with cy3-streptavidin. The signals were detected as described before.

FIG. 30. Detection of cytokine expression from one pair of samples using the two color protein array system. In A), a fixed amount of biotin labeled TNFα was incubated with cy3-streptavidin and an increasing amount of biotin labeled TNFα incubated with cy5-streptavidin in the 1.5 ml centrifuge tubes. Both samples were then mixed together and added to the same human cytokine chip. In B), a fixed amount of biotin labeled TNFα was incubated with cy5-streptavidin and an increasing amount of biotin labeled TNFα incubated with cy5-streptavidin. Both samples were mixed and coincubated with the human cytokine chip. Signals generated by cy3 and cy5 flourescence were scanned using laser scanner with appropriate channels. The intensities of signals were plotted against concentrations of TNFα.

### DETAILED DESCRIPTION OF THE PREFERRED EMBODIMENTS

As used throughout the specification and in the claims; “a” or “an” can mean one or more, depending upon the context in which it is used.

“Analyte” or “analyte molecule” refers to a molecule, typically a macromolecule, such as a polynucleotide or polypeptide, whose presence, amount, and/or identity are to be determined. The analyte is one member of a ligand/anti-ligand pair.

“Antibody,” as used herein, means a polyclonal or monoclonal antibody. Further, the term “antibody” means intact immunoglobulin molecules, chimeric immunoglobulin molecules, or Fab or F(ab′)₂ fragments. Such antibodies and antibody fragments can be produced by techniques well known in the art which include those described in Harlow and Lane (Antibodies: A Laboratory Manual Cold Spring Harbor Laboratory, Cold Spring Harbor, N.Y. (1989)) and Kohler et al. (Nature 256: 495-97 (1975)) and U.S. Pat. Nos. 5,545,806, 5,569,825 and 5,625,126, incorporated herein by reference. Correspondingly, antibodies, as defined herein, also include single chain antibodies (ScFv), comprising linked V_{H} and V_{L} domains and which retain the conformation and specific binding activity of the native idiotype of the antibody. Such single chain antibodies are well known in the art and can be produced by standard methods. (see, e.g., Alvarez et al., Hum. Gene Ther. 8: 229-242 (1997)). The antibodies of the present invention can be of any isotype IgG, IgA, IgD, IgE and IgM.

“Antigen,” as used herein, includes substances that upon administration to a vertebrate are capable of eliciting an immune response, thereby stimulating the production and release of antibodies that bind specifically to the antigen. Antigen, as defined herein, includes molecules and/or moieties that are bound specifically by an antibody to form an antigen/antibody complex. In accordance with the invention, antigens may be, but are not limited to being, peptides, polypeptides, proteins, nucleic acids, DNA, RNA, saccharides, combinations thereof, fractions thereof, or mimetics thereof.

Conditions whereby an antigen/antibody complex can form as well as assays for the detection of the formation of an antigen/antibody complex and quantitation of the detected protein are standard in the art. Such assays can include, but are not limited to, Western blotting, immunoprecipitation, immunofluorescence, immunocytochemistry, immunohistochemistry, fluorescence activated cell sorting (FACS), fluorescence in situ hybridization (FISH), immunomagnetic assays, ELISA, ELISPOT (Coligan, J. E., et al., eds. 1995. Current Protocols in Immunology. Wiley, New York.), agglutination assays, flocculation assays, cell panning, etc., as are well known to the person of skill in the art.

“Bind,” as used herein, means the well understood antigen/antibody binding as well as other nonrandom association between an antigen and an antibody. “Specifically bind,” as used herein describes an antibody or other ligand that does not cross react substantially with any antigen other than the antigen, or antigens, specified. For instance, “specific binding” of an antibody to a class of antigens having an epitope in common is contemplated.

“Capture protein,” as used herein, is an immobilized protein which binds, is bound by, or forms a complex with, one or more analytes of interest in a sample to be tested.

“Detection protein,” as used herein, is a protein comprising a detectable label which binds, is bound by, or forms a complex with, one or more analytes of interest in a sample to be tested or is a protein which binds, is bound by, or forms a complex with, one or more analytes of interest which can be bound by further species that comprise a detectable label. Examples of detectable labels include, but are not limited to, nucleic acid labels, chemically reactive labels, fluorescence labels, enzymic labels and radioactive labels.

“Discrimination antibodies,” as used herein, means antibodies which bind to a specific subset of proteins, wherein binding indicates the presence of some characteristic of any protein bound by the discrimination antibody. The characteristic may be the presence of a specific structure, a specific epitope, a specific sequence or an ability to be bound by a specific antibody, e.g., the discrimination antibody.

“Fusion protein,” as used herein, is a recombinant protein comprising amino acid sequence derived from more than one protein.

“Membrane,” as used herein, means the well understood material of commerce and widespread use in the field of biotechnology, as well as other flexible, non-rigid sheets of polymeric or elastomeric materials. Examples include, but are not limited to, nylon, nitrocellulose, or equivalent materials known to those of skill in the art.

“Microarray,” as used herein, is an ordered arrangement of array elements (e.g., capture proteins) capable of binding other species. The elements are arranged so that there are preferably at least one or more different array elements, more preferably at least 10 array elements, and most preferably at least 100 array elements, and even more preferably 10,000, on a 1 cm² substrate surface.

“Mimetic,” as used herein, includes a chemical compound, or an organic molecule, or any other mimetic, the structure of which is based on or derived from a binding region of an antibody or antigen. For example, one can model predicted chemical structures to mimic the structure of a binding region, such as a binding loop of a peptide. Such modeling can be performed using standard methods. In particular, the crystal structure of peptides and a protein can be determined by X-ray crystallography according to methods well known in the art. Peptides can also be conjugated to longer sequences to facilitate crystallization, when necessary. Then the conformation information derived from the crystal structure can be used to search small molecule databases, which are available in the art, to identify peptide mimetics which would be expected to have the same binding function as the protein (Zhao et al., Nat. Struct. Biol. 2: 1131-1137 (1995)). The mimetics identified by this method can be further characterized as having the same binding function as the originally identified molecule of interest according to the binding assays described herein.

Alternatively, mimetics can also be selected from combinatorial chemical libraries in much the same way that peptides are. (Ostresh et al., Proc. Natl. Acad. Sci. USA 91: 11138-11142 (1994); Dorner et al., Bioorg. Med. Chem. 4: 709-715 (1996); Eichler et al., Med. Res. Rev. 15: 481-96 (1995); Blondelle et al., Biochem. J. 313: 141-147 (1996); Perez-Paya et al., J. Biol. Chem. 271: 4120-6 (1996)).

“Solid support,” as used herein, means the well-understood solid material to which various components of the invention are physically attached, thereby immobilizing the components of the present invention. The term “solid support,” as used herein, means a non-liquid substance. A solid support can be, but is not limited to, a membrane, sheet, gel, glass, plastic or metal. Immobilized components of the invention may be associated with a solid support by covalent bonds and/or via non-covalent attractive forces such as hydrogen bond interactions, hydrophobic attractive forces and ionic forces, for example.

The invention provides arrays useful for the detection, characterization and/or quantitation of antigens or antibodies. The invention further provides methods for the production of arrays of the invention. The invention further provides methods for the use of the arrays of the invention.

The present invention relates to a method of detecting a specified protein. More specifically, a method of detecting a protein that comprises the immobilization of capture proteins onto a membrane to form a microarray. A capture protein of the invention is able to bind to the specified protein, also referred to herein as the protein of interest, thereby “capturing” the protein of interest. The method can further comprise the use of a detection protein. A detection protein is able to bind to the immobilized specified protein. The method further comprises the detection of bound detection protein, thereby indicating the presence of the specified protein. Detection may be accomplished by use of a detectable label attached directly to the detection protein, or detection may be accomplished by use of a detectable label which may, through manipulation known to those of skill in the art, be associated with the detection protein. Examples of the latter include, but are not limited to, the use of a biotinylated detection protein and the a detectable label conjugated to streptavidin or avidin.

In a preferred embodiment, any sample or preparation containing the “protein of interest” can be detectably labeled. For example, a sample containing a protein of interest can be biotinylated and then contacted with the microarray, thereby allowing it to be bound by capture proteins contained in the array. The presence of the protein of interest at a particular location, such as that corresponding to a particular capture agent, can be determined by addition of streptavidin—or avidin—that is conjugated to a detectable label. Other types of detectable labeling of proteins of interest can also be employed, including, but not limited to, Cy3 and Cy5 labels, gold particles, and radiolabels.

In a preferred embodiment, the capture protein is an antigen and the protein detected is an antibody. In a particular further embodiment, the protein detected is a specific antibody. The detection protein can be an antibody. In further embodiments, the detection protein is an anti-antibody and still further, it may be an idiotype-specific anti-antibody.

In a preferred embodiment, the capture protein is an antibody and the protein detected is an antibody. In a particular further embodiment, the protein detected is a specific antibody. In a particular further embodiment, the detection protein is an antibody. In further embodiments, the detection protein is an anti-antibody and still further it may be an idiotype-specific anti-antibody.

In a preferred embodiment of the invention, the capture protein is an antibody and the protein detected is an antigen. In a further embodiment, the protein detected is a specific antigen. In a further embodiment, the detection protein is an antibody. In a further embodiment, the detection protein is an epitope-specific antibody.

In a preferred embodiment of the invention, the capture protein is a protein which is able to bind a protein of interest wherein the capture protein and the protein of interest are not antigen-antibody cognates. For instance, where a first and second protein normally form complexes or interact with one another, the first protein may be immobilized to a membrane so as to act as the capture protein, the second protein may be immobilized by the interaction between the first and second proteins and the presence of the second protein in a sample may be determined by use of a detection protein. In another embodiment of the invention, the capture protein and protein of interest may be such that they normally do not form complexes or interact with one another, but that the conditions or proteins may be altered so as to effect complex formation and thereby effect capture of the protein of interest. It is contemplated that one protein, either the capture protein or the protein of interest can interact with different proteins under different conditions or may interact with many different proteins under identical conditions.

In a preferred embodiment of the invention, the capture proteins are specific for specified proteins and therefore, specifically bind specific proteins. Specific proteins, as defined herein, may mean a single species of protein, or a number of proteins which share common characteristics, such as, but not limited to, common structural motifs, common sequence or an ability to interact with other species in an identical or very similar manner. In a further preferred embodiment, the capture proteins are specific for specified epitopes.

In a preferred embodiment of the invention, the detection proteins are horseradish-peroxidase labeled proteins. In a further embodiment, the detection proteins are horseradish-peroxidase labeled antibodies. In a further embodiment, the detection protein is detected by chemiluminescence. In a further preferred embodiment, the detection protein is detected by enhanced chemiluminescence.

In a preferred embodiment of the invention, the capture proteins bind cytokines. In a further embodiment, the detection proteins bind cytokines. Anti-cytokine antibodies are preferred examples of both capture and detection proteins. Examples of suitable anti-cytokine antibodies include, but are not limited to, anti-human G-CSF, anti-human IL-10, anti-human GM-CSF, anti-human IL-13, anti-human GROα anti-human IL-15, anti-human IFN-γ, anti-human MCP-1 anti-human IL-1α, anti-human MCP-2, anti-human IL-2, biotinylated anti-human MCP-3, anti-human IL-3, biotinylated anti-human MIG, biotinylated anti-human IL-5, biotinylated anti-human/mouse/pig TGF β1, anti-human IL-6, polyclonal rabbit anti-human RANTES, anti-human IL-7, biotinylated anti-human TNF-α, anti-human IL-8, anti-human TNF-β, monoclonal anti-human ENA-78 antibody, monoclonal anti-human I-309 antibody, monoclonal anti-human IL-11 antibody, monoclonal anti-human IL-12 p70, antibody, monoclonal anti-human IL-15 antibody, monoclonal anti-human IL-17 antibody, monoclonal anti-human M-CSF antibody, monoclonal anti-human MDC antibody, monoclonal anti-human MIP-1α antibody, monoclonal anti-human MIP-1β antibody, monoclonal anti-human MIP-1δ/Leukotactin antibody, monoclonal anti-human SCF antibody, monoclonal anti-human/mouse SDF-1 antibody, monoclonal anti-human Tarc antibody and monoclonal anti-human IL-4 antibody.

In further preferred embodiments of the invention, the capture proteins bind growth factor related proteins, angiogenesis or anti-angiogenesis related proteins, particularly secreted angiogenesis factors. In a further embodiment, the detection proteins bind growth factor related proteins, angiogenesis or anti-angiogenesis related proteins, particularly secreted angiogenesis factors. In further embodiments of the invention, the capture proteins and detection proteins are selected from species which bind to infection-associated antibodies or antigens. These antibodies or antigens may be proteins or antigens from the pathogenic species which infects the infected subject, or may be protein, antigens or antibodies elicited in response to infection of a subject.

In a preferred embodiment of the invention, the capture proteins bind more than one specified protein. An example of such a capture protein includes, but is not limited to, an antibody which binds structurally related proteins or an antibody which binds a common, or closely related, amino acid sequence. In a preferred embodiment of the invention, the detection proteins bind more than one specified protein. An example of such a detection protein includes, but is not limited to, an antibody which binds structurally related proteins or an antibody which binds a common, or closely related, amino acid sequence.

In a preferred embodiment of the invention, the specified protein is a fusion protein comprising an engineered epitope and further amino acid sequence. In a further embodiment, the capture protein binds the engineered epitope. In a further embodiment, the detection protein binds the engineered epitope.

The present invention relates to a method for detecting protein. More specifically, the method of detecting protein comprises immobilizing proteins in a microarray on membrane, contacting the microarray with a solution containing discrimination antibodies capable of binding to selected idiotypes to form discrimination complexes, contacting the microarray with a solution containing detection protein capable of binding discrimination complexes and detecting bound detection protein.

In a preferred embodiment of the invention, the detection proteins are antibodies. In a further embodiment, the detection proteins are horseradish-peroxidase labeled antibodies. In a further embodiment, the detection proteins are detected by chemiluminescence and still further, they may be detected by enhanced chemiluminescence. In a further embodiment, the detection protein is an anti-IgA, anti-IgD, anti-IgE, anti-IgG or anti-IgM antibody and the detection protein can bind to more than a single species of discrimination antibody.

In further preferred embodiments, arrays comprising antibodies against proteins of interest can be used to capture proteins and the phosphorylation or modification of the proteins may be determined by detection of phosphorylation by use of detection antibodies specific for phosphorylated amino acids. Examples of suitable anti-phosphorylation antibodies include those raised against phosphotyrosine, phosphoserine or phosphothreonine. Similarly, proteins which specifically recognize carbohydrate moieties, such as concavalin A and wheat germ agglutinin may be used to detect carbohydrate moieties of glycoproteins. Antibodies specific for ubiquitin may be used to detect ubiquitination.

The present invention relates to a method of forming a microarray of proteins on a membrane for use in the practice of other aspects of the invention. More specifically, the method of applying a protein onto defined positions on a membrane, whereby repeated application of different proteins allows the formation of an ordered arrangement of protein array elements. More particularly, the array elements consist of capture proteins capable of binding other species. Each array element may be formed by a single species of capture protein or multiple species of capture proteins. If an array element is formed from multiple species of capture proteins, they may be selected so as to bind a single species of analyte or they may be selected so as to bind multiple species of analytes. An array element formed from multiple species of capture proteins, each selected so as to bind a single species of analyte may be formed from a group of capture proteins which differ from one another in characteristics other than their binding specificity. For example, a multitude of capture proteins which differ in their binding capacity for the analyte at different pH values can be combined to provide an array element which operates effectively over a broader range of pH values. Another example of an array element using multiple capture proteins to capture a single species would be an element comprising multiple antibodies which recognize different epitopes of the same analyte species. Different embodiments of this aspect of the invention which provide array elements comprising capture proteins which bind a single protein of interest over a wide range of environmental conditions are contemplated and are provided herein to the practitioner of skill in the art without undue experimentation.

A capture protein that binds a specific epitope, such as an antibody which specifically binds a particular structural motif that is common to a number of proteins, is contemplated herein. An example of such a capture protein would be the antibody against the carboxy terminus of Mek1 disclosed by Giroux et al. (Current Biology 9: 369-372 (1999)), the antibodies disclosed by Wang et al. (Mol. Cell Biol. 20: 4505-4512 (2000)) or any other antibody identified by one of skill in the art to bind to multiple species. Many such antibodies are known to those of skill in the art and the use of such antibodies is contemplated in the use of the current invention. Furthermore, detection proteins that bind a specific epitope, such as an antibody which specifically binds a particular structural motif that is common to a number of proteins, is also contemplated herein. Such a multispecific detection protein may be used to identify the presence of a specific motif, amino acid sequence or other characteristic in a bound analyte or bound protein of interest. Furthermore, such a multispecific detection protein may be used to characterize immobilized proteins.

In a preferred embodiment of the invention, the membrane is made from polyvinylidene fluoride, nitrocellulose, nylon and/or other suitable materials. Examples of membranes contemplated herein include, but are not limited to, PVDF, Biotrans (ICN), Zeta-probe (Bio-Rad), Colony/Plaque Screen (NEN), Hybond-N⁺ (Amersham), Magnacharge (MSI), Magnagraph (MSI) and Hybond ECL (Amersham). Alternatively, the array of the present invention can be provided on other solid support materials as are known to those of skill in the art, including, but not limited to, polyacrylamide layers on glass or other solids. In a further embodiment, the proteins are antibodies, antigens or are unknown proteins from biological samples. In a further embodiment, each discrete region of the array contains proteins from a selected group of cells, viruses or other protein-containing species and still further, the selected species may be clonal in nature or may be obtained from a tissue sample. In a further embodiment, the proteins are cytokines.

The present invention relates to the microarray formed by the method of forming a microarray of proteins on a solid support. Examples of such solid supports include, but are not limited to, membranes, plastics, gels, sols, glass, ceramics and metal. For a general discussion of microarrays and suitable supports, see Shalon et al. (Genome Research 6: 639-645 (1996), LeGendre (BioTechniques 9: 788-805 (1990)), U.S. Pat. No. 6,197,599 and U.S. Pat. No. 6,140,045, each of which is incorporated herein by reference. In a preferred embodiment of the invention, the microarray is formed on a membrane made from polymeric, elastomeric or other suitable membrane material. Examples of such materials include, but are not limited to, PVDF, nitrocellulose, nylon or modified variants thereof. The current invention contemplates the use of any such material such as is known to those of skill in the art for use in Northern, Southern or Western blotting. Particular aspects of membranes which are desirable to optimize the invention include the ability to bind large amounts of protein, the ability to bind protein with minimal denaturation and the inability to bind proteins that are not proteins of interest when used as described herein. Further, another suitable aspect is that the membrane displays minimal “wicking” when protein solution containing proteins to be used as capture proteins are applied to the membrane. A membrane with minimal wicking allows small aliquots of protein containing solution applied to the membrane to result in small, defined spots of immobilized protein. In contrast, membranes with less suitable wicking characteristics results in larger, more diffuse spots of immobilized protein when applied to the membrane.

Methods of generating the arrays and microarrays of the invention include, but are not limited to, any of the methods commonly used for application of protein solution to a membrane, as well as other methods used to apply a liquid solution to a flexible, solid support (for example, inkjet or bubble jet printing). The use of a pipetman, as described in the examples, is one method for applying a protein solution to a membrane to generate an array. Further examples for the preparation of such protein arrays on membranes, and the use of such protein arrays to detect the presence of specified proteins or analytes is described in U.S. Pat. No. 6,197,599, incorporated herein by reference. Examples of other methods, as one would use to apply a liquid solution to a flexible, solid support, would include the “dot blot” approach. In this method, a vacuum manifold transfers a plurality, e.g., 96, aqueous samples of proteins from 3 millimeter diameter wells to a porous membrane. A common variant of this procedure is a “slot-blot” method in which the wells have highly-elongated oval shapes.

A more efficient technique employed for making ordered arrays of proteins uses an array of pins dipped into the wells, e.g., the 96 wells of a microtiter plate, for transferring an array of samples to a substrate, such as a porous membrane. One array includes pins that are designed to spot a membrane in a staggered fashion, for creating an array of 9216 spots in a 22×22 cm area (Lehrach, et al., Hybridization Fingerprinting in Genome Mapping and Sequencing, Genome Analysis, Vol. 1 (Davies and Tilgham, Eds.), Cold Spring Harbor Press, pp. 39-81 (1990)).

Another method for generating a microarray contemplated involves dispensing a known volume of a reagent at each selected array position, by tapping a capillary dispenser on the support under conditions effective to draw a defined volume of liquid onto the support, wherein this process is repeated using selected reagents at each selected array position to create a complete array. The method may be practiced in forming a plurality of such arrays, where the solution-depositing step is applied to a selected position on each of a plurality of solid supports at each repeat cycle. Further description of such a method may be found in U.S. Pat. No. 5,807,522, incorporated herein by reference.

In a further contemplated method, devices normally utilized for printing on paper can be utilized to generate the microarrays of the invention. For example, the desired reagent can be loaded into the printhead of a desktop jet printer and printed onto suitable membranes. Silzel et al. (Clinical Chemistry 44: 2036-2043 (1998)), incorporated herein by reference, describes the use of a similar method to generate a multianalyte array on a solid support.

In a preferred embodiment of the invention, the microarray generated on the membrane has a density of at least 5 spots/cm², preferably at least 10, 20, 30, 40, 50, 60, 70, 80, 90, 100, 110, 120, 130, 140, 150, 160, 170, 180, 190, 200, 210, 220, 230, 250, 275, 300, 325, 350, 375, 400, 425, 450, 475, 500, 550, 600, 650, 700, 750, 800, 850, 900, 950, 1000, 2000, 3000, 4000, 5000, 6000, 7000, 8000 or 9000 spots/cm² and most preferably at least 10,000 spots/cm².

It is contemplated that the spots on the membrane may each represent a different species of protein or that the multiple spots on the membrane may represent the same species of protein. In a further embodiment, the spots each represent an array element of differing identity or characteristics.

The present invention also relates to a method of forming a microarray of proteins using a first and second binding partner wherein each discrete region in the microarray has a selected composition, character or identity. More specifically, the method relates to the formation of a microarray wherein a first binding partner, which can bind a second binding partner, is immobilized on a membrane and a second binding partner, or second binding partners, is applied to the membrane under conditions wherein the first and second binding partners form a stable complex, thereby immobilizing the second binding partner. As a benefit of this aspect of the invention, the first binding partner can be selected so as to maximize the binding of the second binding partner, thereby increasing the effective density of second binding partner on the membrane. This increased density allows the use of smaller array elements to achieve an equivalent signal. Further, selected first binding partners may be chosen so as to effectively block the non-specific binding of sample of the membrane, thereby increasing the sensitivity of each array element by reducing the level of background signal. The combined effects of the improvements in capture protein density and in reduced background allow the effective use of microarrays of significantly higher density.

In a preferred embodiment of the invention, the first binding partner is a protein. In accordance with the invention, the first binding partner may be applied to selected portions of the microarray or may be used to coat the entire membrane. In a further embodiment, the first binding partner is an idiotype-specific antibody and still further, the second binding partner may be an antibody containing the idiotype. A particularly preferred embodiment is that wherein the first binding partner specifically orients the second binding partner so as to maximize its effectiveness as a capture protein, for example, the first binding partner may be a monoclonal antibody specific for an epitope of capture antibodies that positions the capture antibodies onto the membrane in an optimal spatial orientation for binding a protein of interest.

The binding partners described herein are not limited to being proteins or antibodies. In other preferred embodiments of the invention, the first binding partner can be biotin or a biotinylated protein and the second binding partner is avidin, streptavidin or comprises portions thereof or the first binding partner can be avidin, streptavidin or comprise portions thereof and the second binding partner is biotin or a biotinylated protein.

In a preferred embodiment of the invention, the second binding partner is a fusion protein comprising a first amino acid sequence whose presence allows binding to a first binding partner and a second amino acid sequence whose presence allows binding to at least one other protein such that the second binding protein can be a capture protein.

The present invention relates to the microarray formed by the use of any embodiment of the method whereby a first and second binding partner is used to structure an microarray, orient elements of the microarray for greater efficiency or effectiveness or wherein the use of the first and second binding partners increases the density of the microarray and the sensitivity of the microarray-based assay.

The present invention relates to a method of characterizing proteins which relies on binding interactions between proteins. Specifically, the method comprises immobilizing proteins onto a solid support to form a microarray of immobilized proteins, contacting the immobilized proteins of the microarray with detection proteins of known characteristics and detecting binding of detection proteins to the immobilized proteins, thereby determining characteristics of the immobilized proteins. In preferred embodiments, the solid support can be a membrane and the detection proteins can be antibodies or antigens. In a particular embodiment of the invention, the proteins immobilized on the membranes are unknown proteins and the detection proteins are used to characterize and/or identify the unknown proteins. However, it is not necessary that the proteins bound to the membrane be unknown proteins for this aspect of the invention to provide a benefit. In another useful embodiment of the invention, known proteins may be immobilized on the membranes and the binding of known detection proteins may be used to characterize properties of the known proteins immobilized in the microarray. For instance, a multitude of known proteins could be immobilized in a microarray and probed with antibodies specific for certain modifications, such as glycosylation, phosphorylation and ubiquitination, or probed with antibodies specific for certain structural motifs, whereby binding of the selected detection protein indicates the presence of the modification or structural motif in the known, but not entirely characterized, immobilized proteins of the microarray.

In a further embodiment of the present invention, the microarrays are microarrays of immobilized nucleic acids. In this embodiment of the invention, immobilized nucleic acids act as capture nucleic acids, in a manner analogous to capture proteins, to immobilize species of interest, which are then detected using detection proteins. In a particular embodiment, a nucleic acid array is contacted with transcription factors. Transcription factors, and the sequences to which they bind, are detected by detecting the presence of protein at the position of the specific sequences, and by identifying the transcription factors. Immobilized transcription factors can be identified either through the sequence to which they bind, wherein the sequence must be known or determined, by use of detection antibodies, specific for the transcription factor to be identified, or by other methods known to those of skill in the art.

In a further embodiment, the present invention relates to a method of detecting a specified protein. More specifically, a method of detecting a specified protein that comprises immobilizing capture proteins in a microarray on a membrane, labeling the proteins contained in a solution that contains the specified protein, passing the solution containing the specified labeled protein over the microarray of capture proteins and detecting bound specified protein. As described earlier, capture proteins are able to bind to the specified protein. Also, as the immobilized capture proteins can be immobilized to known positions and can be of known binding characteristics, the presence of the protein of interest, the specified protein, can be determined by detection of protein at a site on the microarray that corresponds to a capture protein that binds the protein of interest. Further, the capture protein can be a capture protein that captures a specific protein. Further, the labeling used to label the proteins in the solution, including the specified protein, can be biotinylation. If the labeling used is biotinylation, use of detectably labeled avidin or streptavidin can be used to generate a detectable signal.

The present invention will be illustrated in further detail in the following non-limiting examples.

### EXAMPLE 1

### Simultaneous Detection of Multiple Proteins with an Array-Based Enzyme-Linked Immunosorbant Assay (ELISA) and Enhanced Chemiluminescence (ECL)

Our goal in this example was to test the feasibility of simultaneously and specifically detecting numerous proteins using a 96-well based ELISA coupled with enhanced chemiluminescence (ECL) without the use of any expensive machinery. We demonstrated the potential use of this method to detect numerous proteins simultaneously in a general laboratory setting.

Materials and Methods. Antigens and their corresponding horseradish peroxidase (HRP)-conjugated monoclonal antibodies were purchased from several different companies as shown in Table 1. Antigens and antibodies were prepared as stock solutions (4 mg/ml). Antigens were diluted with TBS (0.01 M Tris HCl pH7.6/0.15 M NaCl) to 5 μg/ml as working solutions prior to experiments. One hundred μl of antigens were immobilized onto polyvinylidine difloride (PDVF) membrane (Immobilon, Millipore, Bedford, Mass., USA) using a 96 well format Bio-Dot apparatus from Bio-Rad (Hercules, Calif., USA). Each well was rinsed with 2×1 ml TBS and vacuum kept for 3-6 min. The membranes were then blocked with 5% bovine serum albumin (BSA) in TBS for 1 hour at room temperature. After blocking, the membranes were incubated with appropriate HRP-conjugated antibodies for 1 to 2 hours at room temperature. The excess antibodies were then removed from the membrane by washing three times with TBS/0.1% Tween 20 (5 min each) and then twice with TBS (5 min each). The membranes were then placed in a plastic box and incubated with ECL substrate according to the manufacturer's instructions (Amersham, Aylesbury, UK). Finally the membranes were placed on a sheet of Kodak X-OMAT film (Eastman Kodak, Rochester, N.Y., USA) for development by autoradiography.

For detection of cytokines, a Sandwich ELISA approach was adapted. Each pair of antibodies was purchased from BD PharMingen (San Diego, Calif., USA). All cytokines, except growth-regulated oncogene α (GROα), were obtained from Peptotech (Rochy Hill, N.J., USA). GROα was the product of BD PharMingen. The capture antibodies were immobilized onto PDVF membranes (Immobilon, Millipore, Bedford, Mass.). The membranes were then incubated with cytokines, either individually or collectively, with other controls or with conditioned media. After extensive washing to remove unbound species (three times with TBS/0.1% Tween 20 and twice with TBS), the membranes were incubated with corresponding detection antibodies. Extensive washing was then carried out again. The membranes were then incubated with 20,000 fold diluted HRP-conjugated streptavidin (BD PharMingen, San Diego, Calif., USA) and the pattern of detection antibody binding was determined by development of the blot according to the manufacturer's instructions for ECL Western blotting detection reagents, incorporated herein by reference (Amersham, Aylesbury, UK).

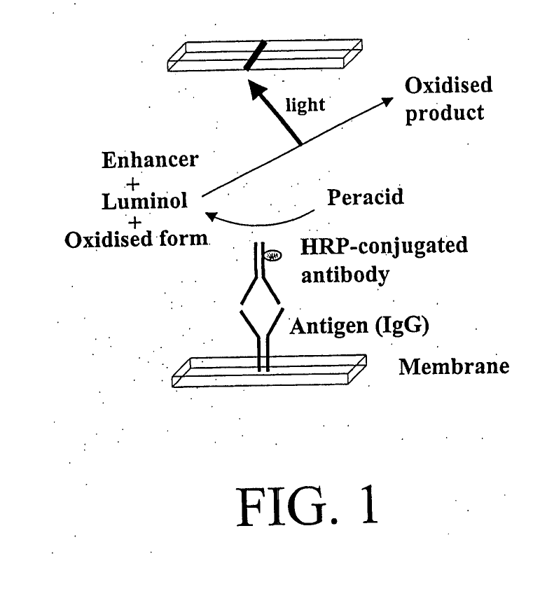

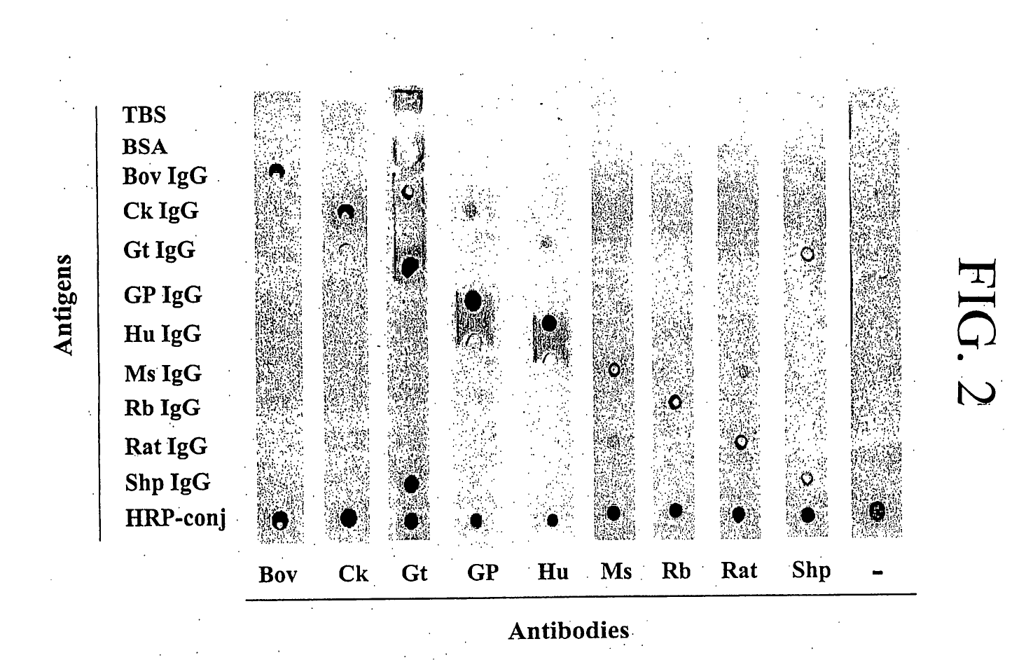

Results. To develop a feasible method, which can simultaneously detect multiple antibodies or proteins and be executed in any laboratory setting, I took the advantage of sensitivity of ECL and specificity of ELISA. The principle of this approach is outlined in FIG. 1. In this particular example, different known specific immunoglobulin Gs (IgGs) were immobilized onto PDVF membranes using a 96 well apparatus. After blocking nonspecific binding sites with BSA, the membranes are incubated with lysate or other samples containing antigens of interest to be detected. After extensive washing to remove nonspecific binding, the membranes are incubated with HRP-conjugated antibodies specific to corresponding antigens. The signals are visualized with ECL.

We first tested the feasibility of this method by using a simplified system. Several different antigens were immobilized onto PDVF membranes through 96 well apparatus. The membranes were then blocked with BSA and different monoclonal antibodies were applied to test the specificity of the assay and to determine the ability to detect multiple analytes at the same time. The membranes included two negative controls (buffer only and BSA) and one positive control (HRP-conjugated antibody). To demonstrate the specificity of each HRP-conjugated monoclonal antibody for its cognate antigen, each row (12 wells) was incubated after a blocking step, with its cognate monoclonal antibody as outlined in Table 1. The antigen-antibody complexes were then detected using an ECL kit. The signals were then visualized by exposure to X-ray film. As shown in FIG. 2, specific signals for cognate antigen-antibody pairs were detected using anti-bovine, anti-chicken, anti-guinea pig, anti-human, anti-mouse, anti-rabbit and anti-rat monoclonal antibodies for their respective antigens. However, with anti-goat (Gt) antibody was strongly cross-reactive with sheep monoclonal antibody (Shp IgG).

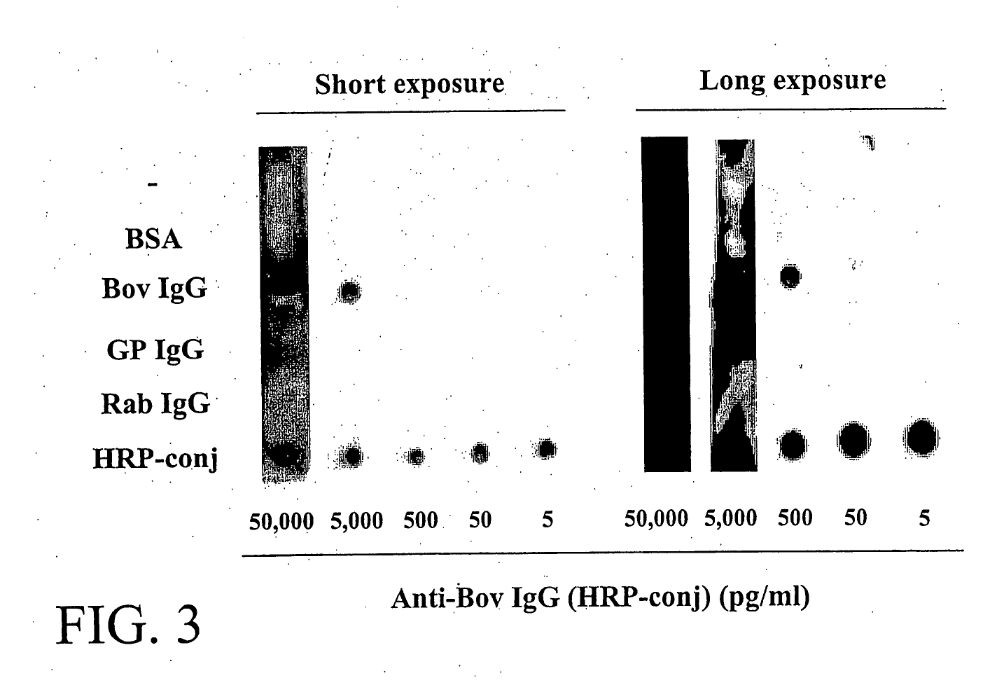

Next the sensitivity of this method was examined. Different IgGs were immobilized onto PDVF membranes as described previously. The membranes were then incubated with different concentrations of HRP-conjugated anti-Bovine IgG. As shown in FIG. 3, antibodies could be detected at concentrations as low as 500 pg/ml to 50 pg/ml.

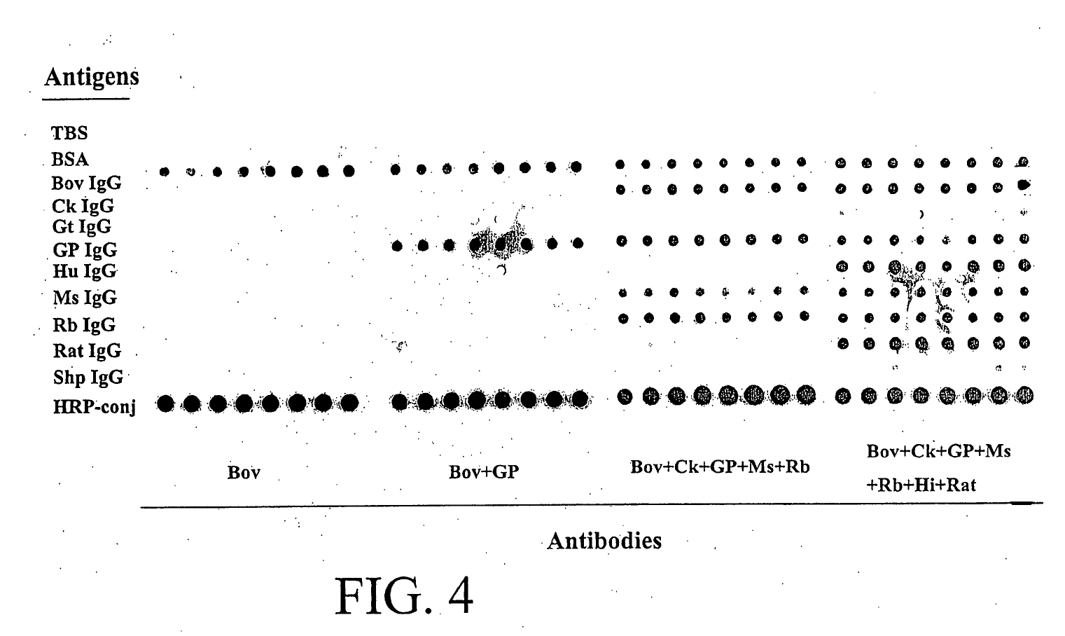

To demonstrate that this approach can detect many different antibodies at the same time, whole membranes (96 wells) were incubated with an individual antibody (Bovine) or combinations of different antibodies (Bovine+Guinea Pig, Bovine+Chicken+Guinea Pig+Mouse+Rabbit and Bovine+Chicken+Guinea Pig+Mouse+Rabbit+Human+Rat). As shown in FIG. 4, multiple antibodies can be detected in 96 well format simultaneously.

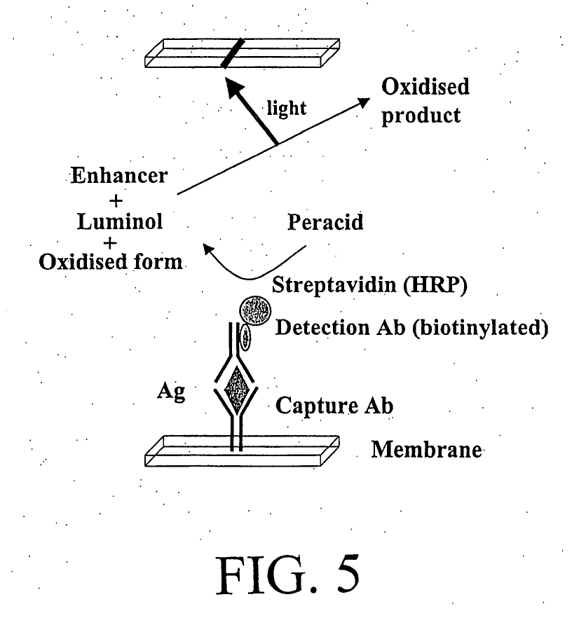

After demonstrating the feasibility and sensitivity of detection of antibodies in the simplified system described above, the methodology was used to test whether it is possible to directly detect antigens in a Sandwich ELISA format. In this approach, a pair of antibodies, which recognize two different epitopes of same antigen, are used. One antibody is immobilized onto PDVF membrane and functions as a capture protein. A second antibody is labeled with biotin and functions as a detection protein. The binding of the detection antibody, thereby detecting the presence of a bound antigen, is detected by use of an ECL system (FIG. 5).

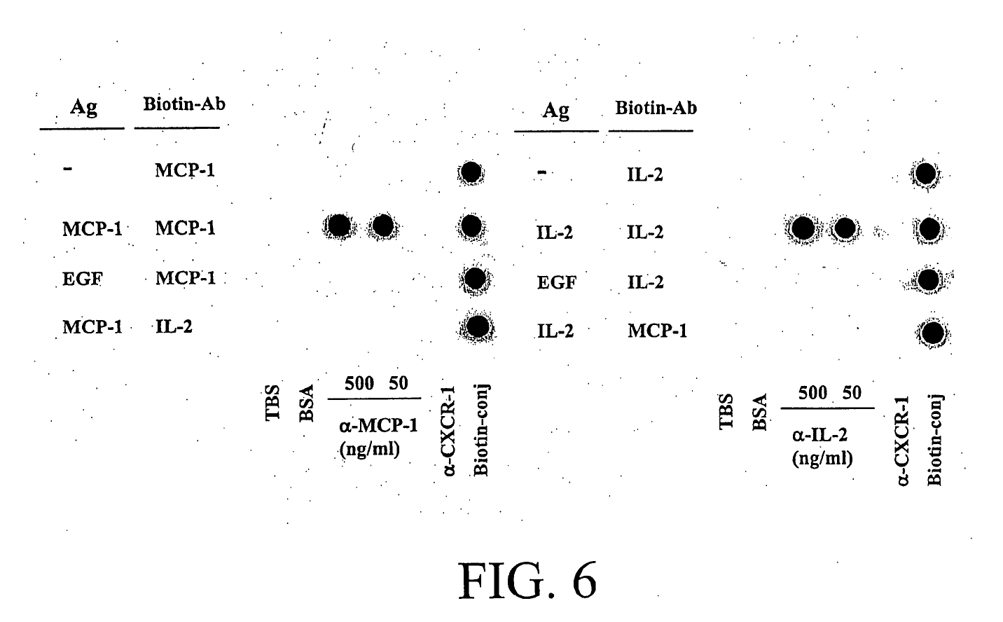

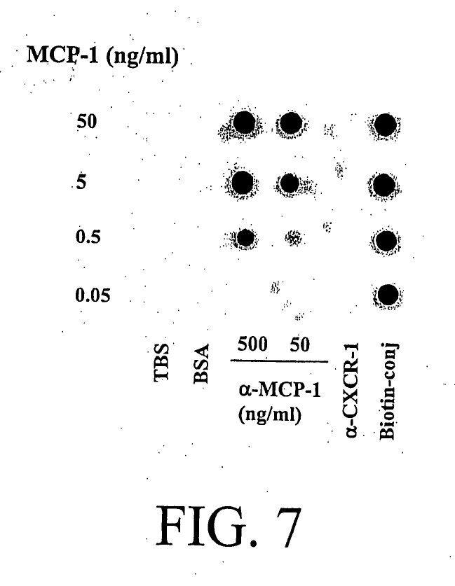

Monocyte chemotactic protein-1 (MCP-1) and Interleukin-2 (IL-2) were used in the model system. Anti-MCP-1 antibody or anti-IL-2 antibody (capture) or other controls were then immobilized onto PDVF membranes as previously described for immobilization of other proteins. The membranes were then incubated with MCP-1 or IL-2 or different negative controls respectively. The membranes were then incubated with biotin-conjugated anti-MCP-1 or biotin-conjugated anti-IL-2 or other negative controls. As shown in FIG. 6, MCP-1 and IL-2 are specifically detected. The sensitivity of detection of MCP-1 was such that concentrations as low as 500 pg/ml of MCP-1 were detected (FIG. 7). The specificity of detection was such that no signal was detected in the control spots where α-CXCR-1, a receptor for CXC class of chemokines, was immobilized.

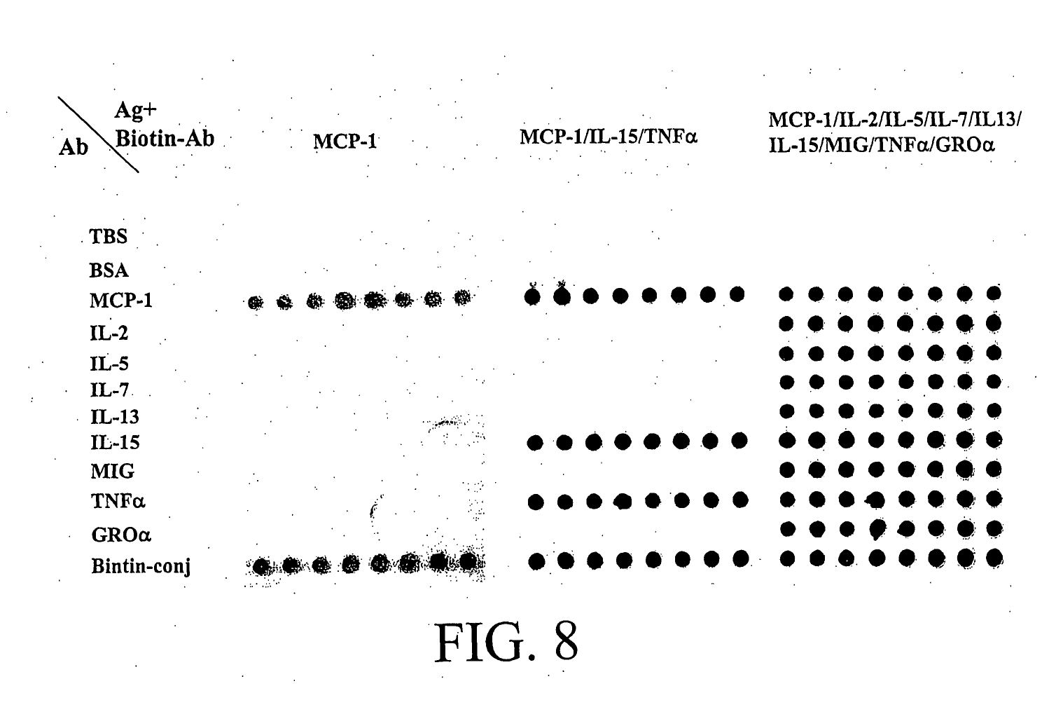

Multiple cytokines can be detected simultaneously using this sandwich assay. Membranes with different capture antibodies immobilized on them were incubated with different combinations of cytokines and their corresponding detection antibodies, in this case, biotin-conjugated antibodies. The specific signals were detected as expected as shown in FIG. 8.

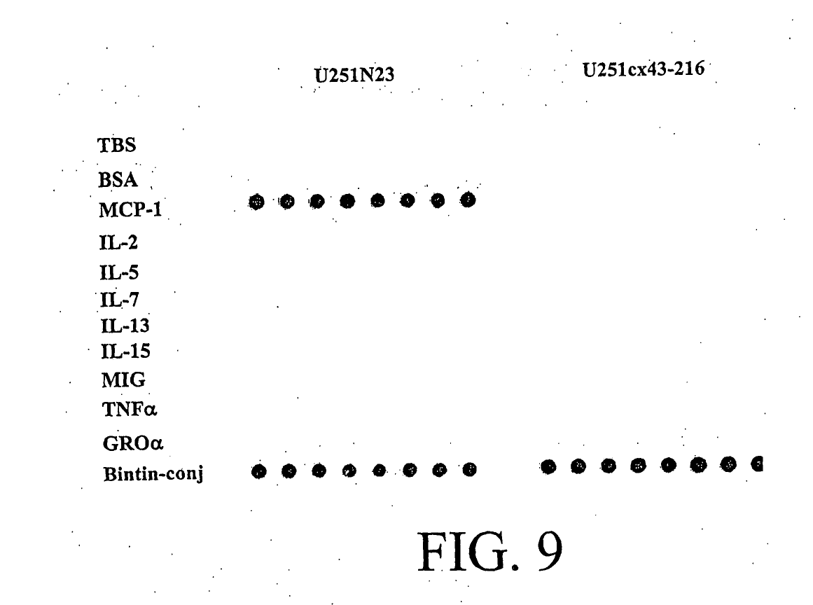

One of the most challenging questions in antibody-based microarrays is whether it can be used to directly detect protein expression product from conditioned media, or patient specimen or crude cell lysates. The conditioned media from cx43 transfected human glioblastoma U251 (U251cx43-216) and control transfected cells (U215N23) (Huang et al., Cancer Res. 58: 5089-5096 (1998)) were collected and incubated with anti-cytokine immobilized membranes. Our unpublished data showed that transfected cx43 profoundly reduced the expression of MCP-1 as demonstrated by cDNA microarray, RT-PCR and immunoprecipitation. As shown in FIG. 9, conditioned medium from N23 showed specific signal to MCP-1, consistent with our previous finding, and indicates that the approach described here can be used to detect protein from biological samples.

### EXAMPLE 2

### Simultaneous Detection of Multiple Cytokines and Antibodies

To simultaneously detect multiple proteins and antibodies, a microspot approach was developed. In this approach, capture proteins, either antibodies or antigens, are spotted onto a membrane. The membrane is then exposed to a sample containing a protein, or proteins, of interest. The protein of interest is bound by its cognate, either an antibody or antigen, spotted onto membrane, is thereby immobilized, and its presence is determined by the binding of a detection protein, specifically an HRP-labeled antibody. Detection of the HRP-labeled antibody's presence by enhanced chemiluminescence indicates the presence of the bound antigen of interest, thereby indicating its presence in the sample.

As a first step, a simplified system was applied to test the feasibility of this assay. Various known specific immunoglobulins (Igs) were spotted onto membrane and detected by incubation with HRP-conjugated antibodies specific to corresponding antigens. The signals were then visualized by ECL.

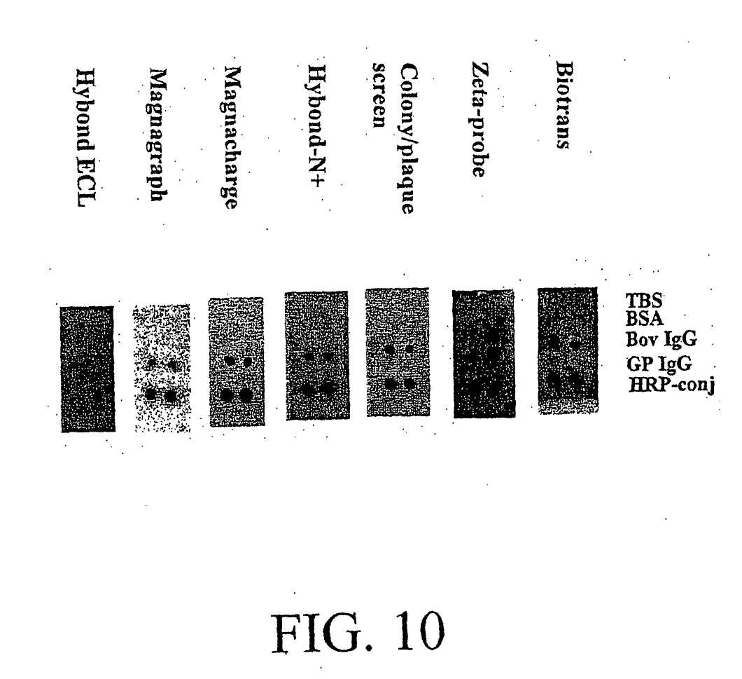

To select a suitable membrane for the assay, bovine IgG and other controls were spotted onto a number of commercially available membranes (Table 2). These membranes were then incubated with HRP-conjugated anti-bovine IgG. As shown in FIG. 10, Magnagraph and Magnacharge resulted in the lowest levels of background signal obtained in the test. As Magnagraph has a higher absorption capacity than Magnacharge, Magnagraph was chosen for use in further assays.

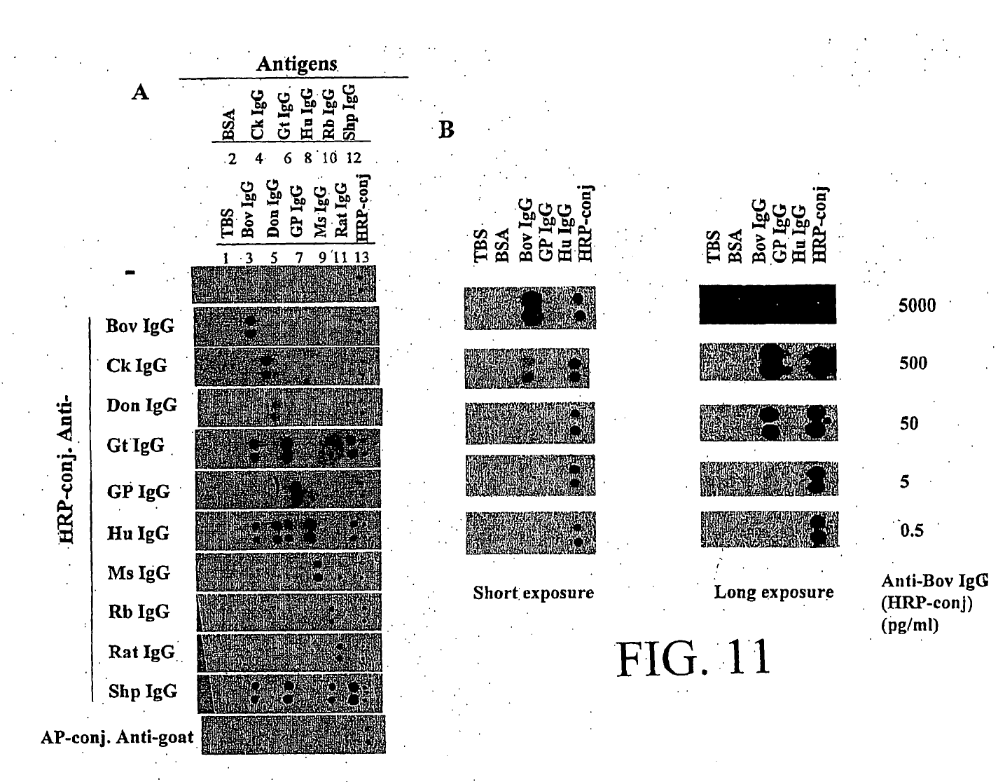

The specificity of the assay was then tested. Different monoclonal antibodies were incubated with membrane spotted with selected IgGs. As shown in FIG. 11A, very low background levels were consistently seen in all cases. Specific recognition of antigen by cognate antibody was also observed for bovine IgG, chicken IgG, donkey IgG, goat IgG, guinea pig IgG, mouse IgG, rabbit IgG and rat IgG. Cross-reactions were noticed among goat IgG, human IgG and sheep IgG.

The sensitivity of the array was demonstrated by incubation of a membrane with different antigens and controls spotted on it with different concentrations of HRP-conjugated anti-bovine IgG. As shown in FIG. 11B, concentrations of HRP-conjugated antibody (anti-bovine IgG) as low as 5 pg/ml of can be detected. Similarly high sensitivity was also seen in other HRP-conjugated antibodies.

Methods

Preparation of higher density array membranes. An array of 504 spots (28×18) on a 6×8 cm membrane was generated using a template to guide manual spotting onto a membrane. Immunoglobulins (IgGs) and corresponding Horseradish peroxidase (HRP)-conjugated monoclonal antibodies were diluted with TBS to 100 μg/ml as working solutions prior to experiments. From the working solutions of antibodies, 0.25 μl of each solution was manually spotted by a 2 μl pipetman. HRP-conjugated or biotin-conjugated antibodies were spotted onto membranes as positive controls and for orientation of arrays during analysis.

Array assay of different species of HRP-conjugated IgGs. Different IgGs (0.25 μl of 100 μg/ml) were loaded onto membranes as described above. Membranes were blocked with 5% BSA (Bovine Serum Albumin)/TBS (0.01 M Tris HCl pH7.6/0.15 M NaCl) for 1 hour at room temperature and incubated individually or collectively with HRP-conjugated antibodies for 2 hours at room temperature. Arrays were then washed three times with TBS/0.1% Tween 20 and then twice with TBS. The presence of HRP-conjugated antibodies, and therefore, the presence of the specified IgGs, was determined by enhanced chemiluminescence as described previously.

Array assay of multiple cytokines. Pairs of antibodies that recognize different epitopes of the same antigen were used to capture and detect a certain antigen. In this example, 0.25 μl of an individual capture protein, an antibody, at a concentration of 200 μg/ml was spotted onto membranes as described above. After blocking with 5% BSA/TBS, membranes were incubated with one or more cytokines prepared in 5% BSA/TBS for 2 hours at room temperature. Unbound cytokines were removed from the membrane by washing with TBS/Tween 20 and TBS. The presence of bound cytokines was detected by binding of biotin-conjugated anti-cytokine antibodies to the bound cytokine. Washing the membranes removed the biotin-conjugated anti-cytokine antibodies. The presence of bound biotin-conjugated anti-cytokine antibodies, and therefore, the presence of bound cytokines, was determined by enhanced chemiluminescence as described previously.

Array assay of multiple antibodies. Different species of IgGs (100 μg/ml) were immobilized onto membranes at a quantity of 0.25 μl per spot as described above to generate an array. Each array was then incubated with various Donkey anti-IgGs either individually or collectively, after blocking with 5% BSA/TBS. Following wash steps, membranes were incubated with rabbit anti-Donkey IgG, which was pre-absorbed with agarose-immobilized guinea pig IgG, goat IgG, human IgG and sheep IgG, to remove cross-reaction components. Imaging of bound donkey anti-IgGs was carried out with enhanced chemiluminescence.

Results

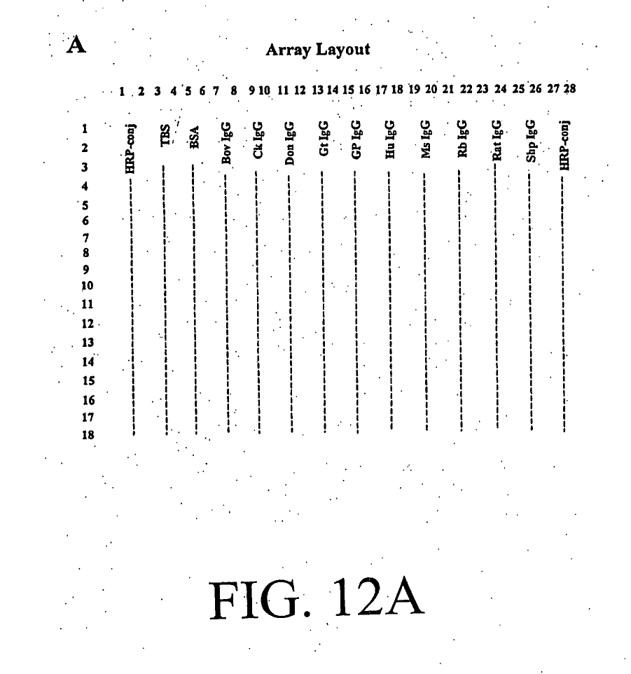

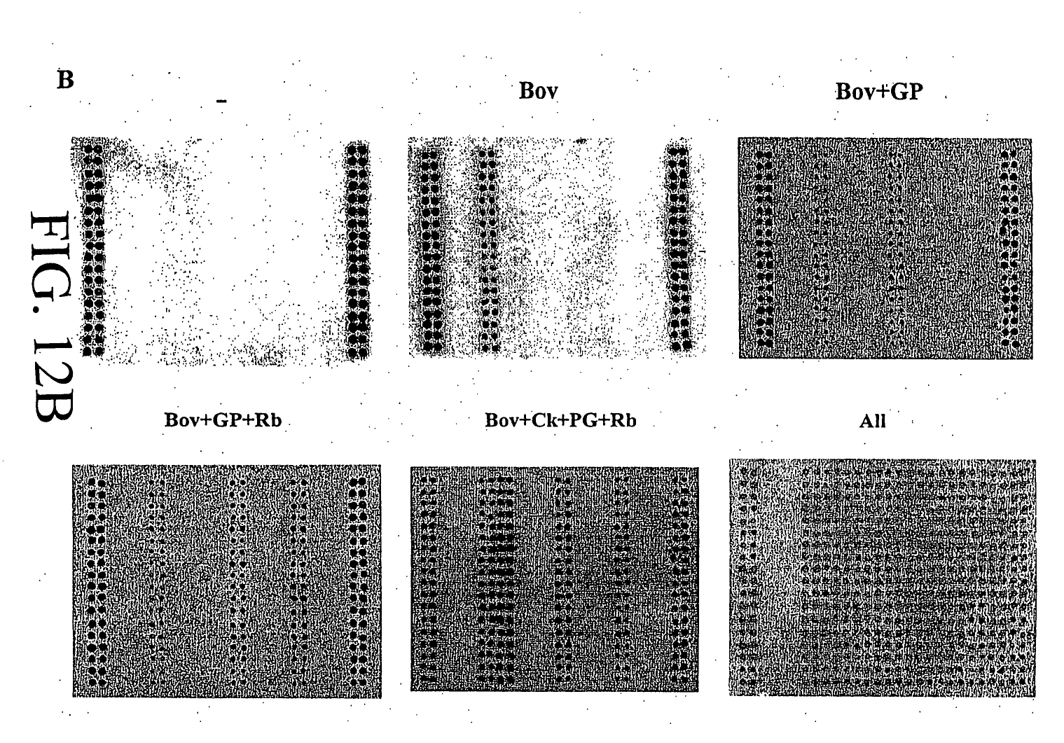

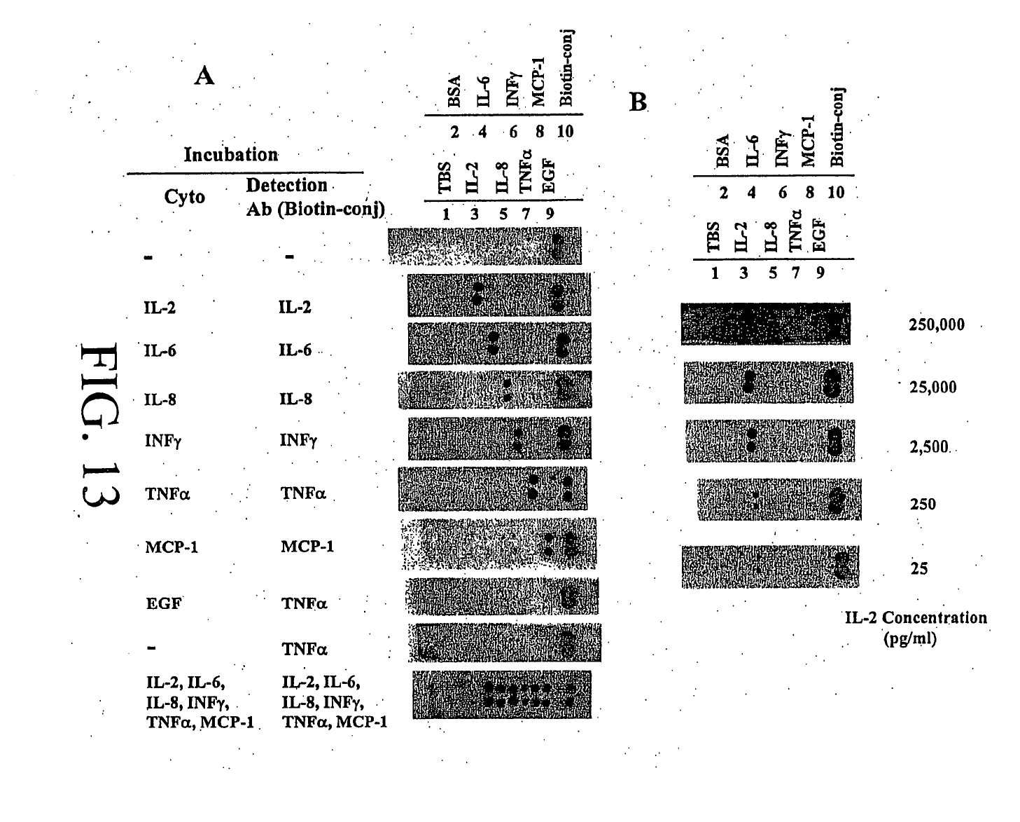

Array assay of different species of HRP-conjugated IgGs. To demonstrate that this approach can be used in high density array format, membranes with a total of 504 spots immobilized with different IgGs as indicated in FIG. 12A were incubated with single or combination of HRP-conjugated antibodies. As shown in FIG. 12B, a total of 504 spots can be simultaneously detected with similar specificity and sensitivity as lower density arrays. Procedures for preparation of membranes and detection of bound signal were as described above.

Array assay of multiple cytokines. The protein array system described above was extended to assay human cytokines. The principle of this assay is based upon the sandwich ELISA and utilizes ECL for detecting antigen binding. Pairs of antibodies, which recognize two different epitopes of same antigen, are used. One antibody is spotted onto membrane and served as a capture protein. The second corresponding antibody is labeled with biotin and served as a detection protein. The binding of the detection protein was detected by use of ECL as described previously.

Different types of membranes, listed in Table 2, were screened for their suitability for use in this assay. Of those screened, Hybond ECL membrane showed the highest sensitivity and lowest background. Consequently, Hybond ECL membrane was used in the assay and analysis of cytokines. Six cytokines were assayed using this array format. The specificity of this assay was first demonstrated with individual cytokines. As shown in FIG. 13, the assay exhibited great specificity for each of the cytokines tested, consistent with ELISA data. All six cytokines were specifically recognized, and bound, by their corresponding capture antibodies and were specifically detected by their cognate detection antibodies. No cross-reaction was observed among the six cytokines or the controls, including EGF, BSA and buffer only. When all six cytokines were simultaneously presented to the array as a mixture, specific signals were detected in the spots where capture antibodies immobilized the six cytokines, but no signal resulted at positions where EGF, BSA or buffer only were spotted onto the membrane. Additional controls further demonstrated the high specificity of this assay, including incubation of membranes with EGF, followed by incubation with an unrelated detection antibody or incubation with detection antibody alone. In addition to being highly specific, this assay was also shown to be highly sensitive. For example, the level of detection possible for IL-2 is lower than 25 pg/ml (FIG. 13B). Several other cytokines, including MCP-1 and TNFα, can also be detected at similarly low concentrations.

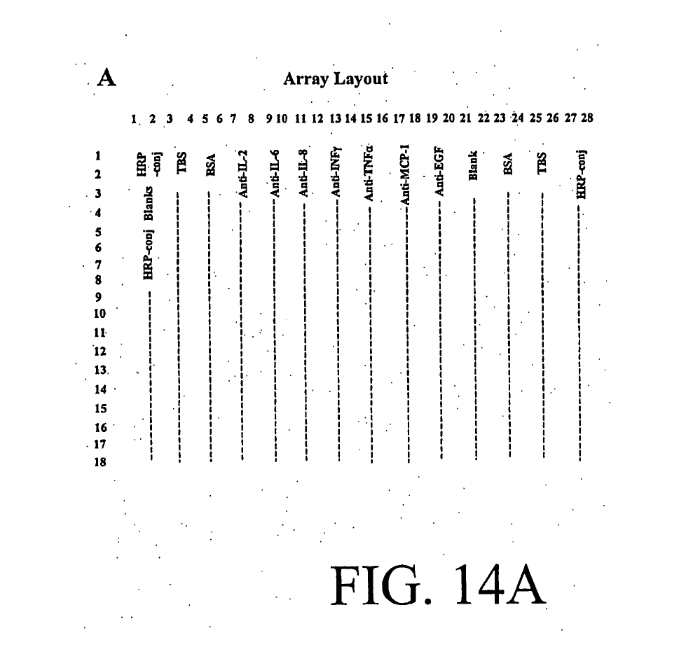

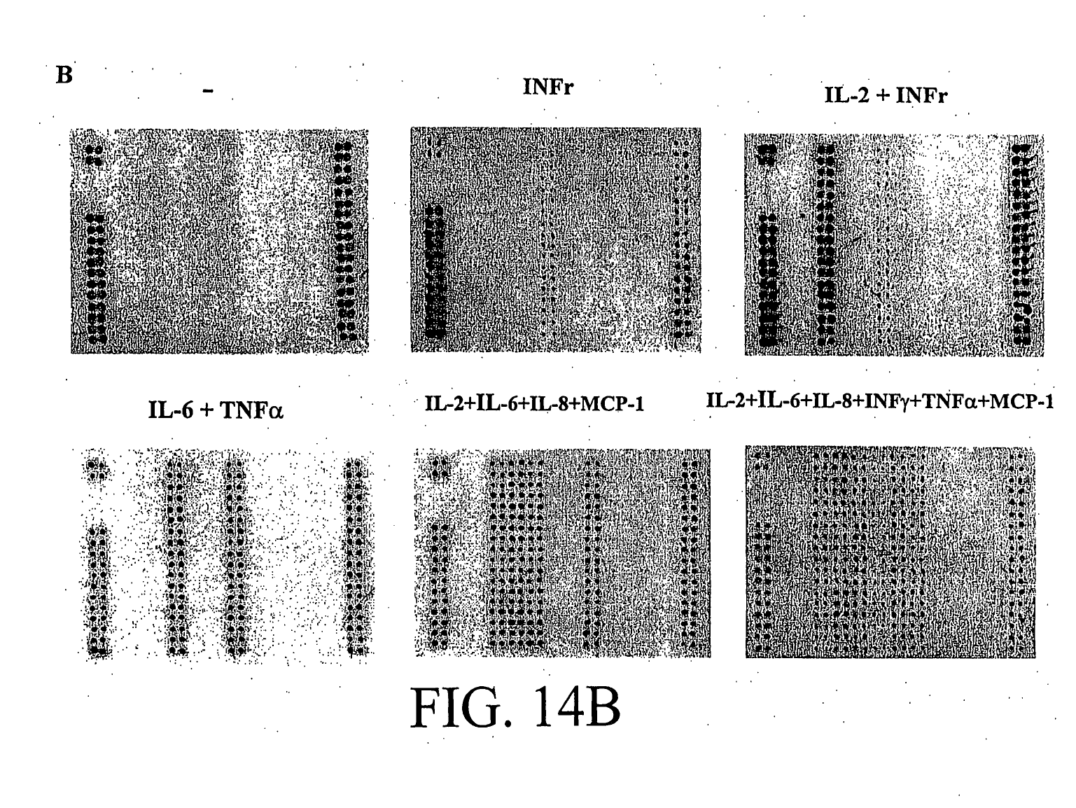

Another example of this approach being used in a high density array format is shown in FIG. 14. In this case, anti-cytokine antibodies and controls were spotted on the membranes to act as capture proteins (FIG. 14A). These membranes were then incubated with a control, INFγ, IL-2 and INFγ, IL-6 and TNFα, IL-2, IL-6, IL-8 and MCP-1, or IL-2, IL-6, IL-8, INFγ, TNFα and MCP-1. Specific binding and detection of the bound antigens can be seen in FIG. 14B.

Array assay of multiple antibodies. The protein array system also can be used to detect antibodies. In this variation of the assay, different known antigens are spotted onto membranes. The membranes are then incubated with samples containing antibodies to be detected. After extensive washing to remove unbound antibodies, the membranes are incubated with HRP-conjugated antibody(ies) against species-specific IgGs, thereby detecting the presence of bound antibodies. The detectable signals from the HRP-conjugated antibodies are then analyzed by use of the ECL methodology as described above.

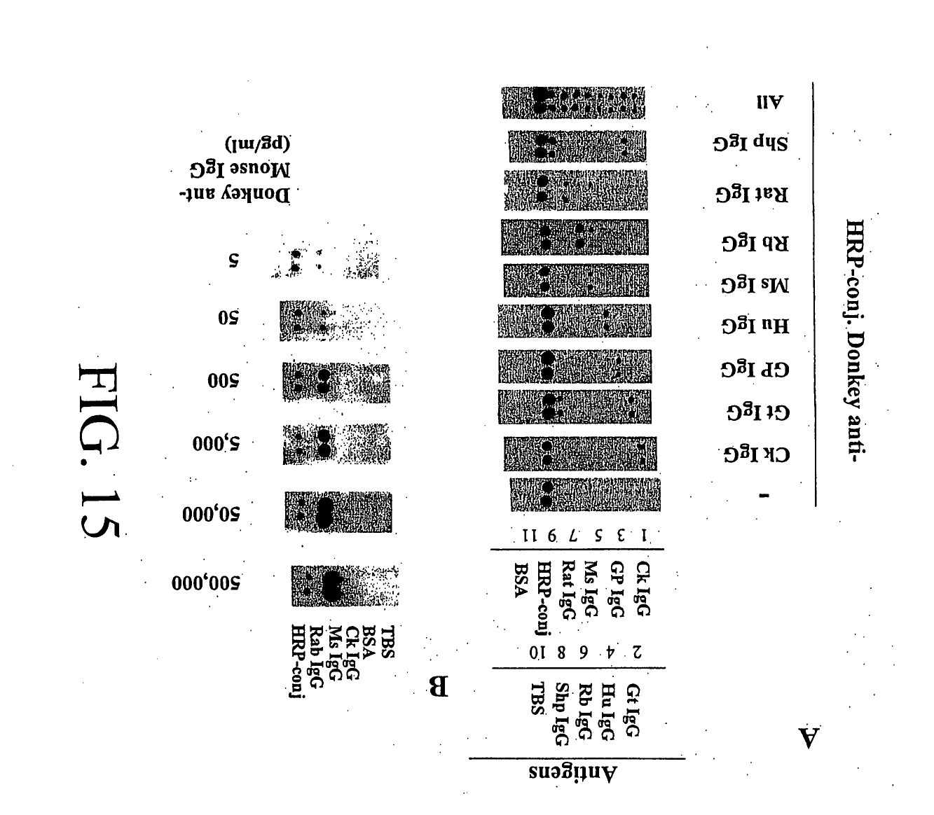

To demonstrate that this is a feasible approach to detect multiple antibodies simultaneously, different species of IgGs were spotted onto the MSI Magnagraph membrane. In this particular example, the IgGs above are the antigens of the antigen/antibody complex and act as the capture proteins. After blocking with BSA, membranes were incubated with individual donkey anti-IgGs against given species. After extensive washing to remove antibodies exhibiting unspecific binding, membranes were incubated with anti-donkey IgG, the detection protein in this case, to detect the presence of bound donkey anti-IgGs against given species, the proteins of interest. Extensive washing was then repeated and the pattern of antibody binding was determined using ECL. As shown in FIG. 15A, specific signals were detected using donkey anti-chicken, anti-guinea pig, anti-human, anti-mouse, anti-rabbit and anti-rat. Some cross-reactivity was observed between donkey anti-goat and anti-sheep. This assay is able to detect the presence of the protein of interest bound to a capture protein at very low concentration. As indicated in FIG. 15B, the concentration of donkey anti-mouse IgG, can be as low as between 5 and 50 pg/ml.

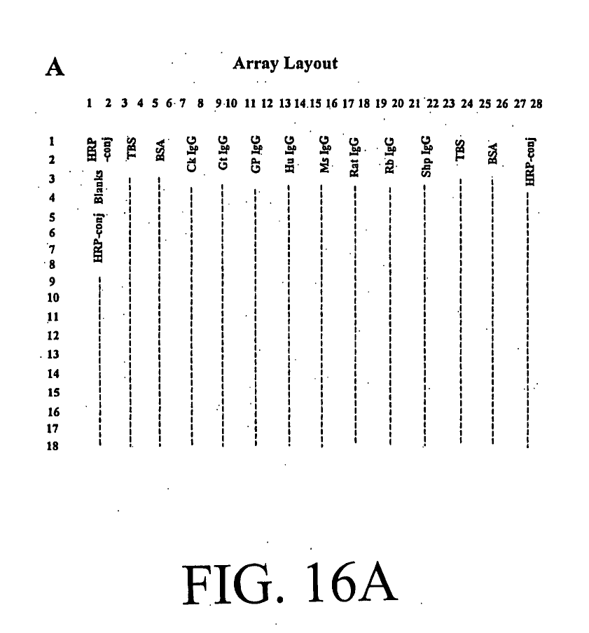

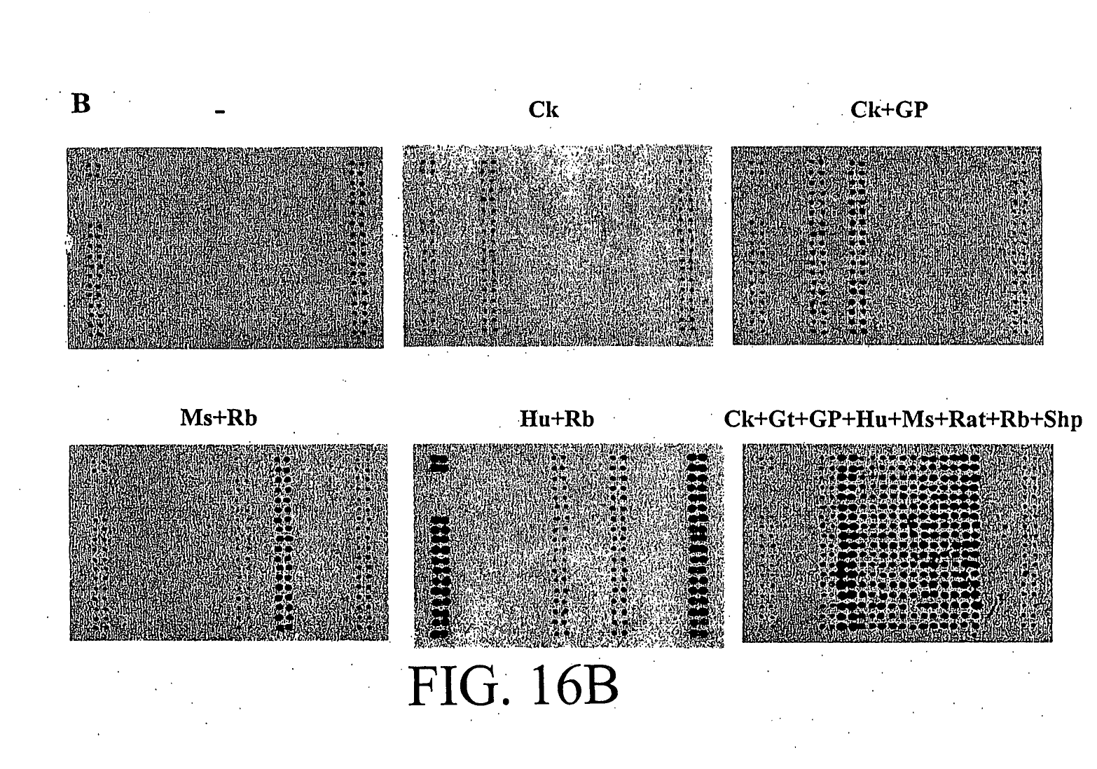

Another example of this approach being used in a high density array format is shown in FIG. 16. In this case, IgGs from various species and controls were spotted on the membranes to act as capture proteins (FIG. 16A). These membranes were then incubated with a control or various combinations of donkey antibodies against the selected IgGs (anti-chicken IgG, anti-chicken IgG and anti-guinea pig IgG, anti-mouse IgG and anti-rabbit IgG, anti-human IgG and anti-rabbit IgG or anti-chicken IgG, anti-goat IgG, anti-guinea pig IgG, anti-human IgG, anti-mouse, IgG, anti-rat IgG, anti-rabbit IgG and anti-sheep IgG). Specific binding and detection of the bound antibodies of interest can be seen in FIG. 16B.

### EXAMPLE 3

### Detection of Multiple Cytokines from Conditioned Media and Patient's Sera

This example describes a highly sensitive ELISA-based protein array system in which multiple cytokines can be simultaneously detected from the experimental model system, from tissue culture media and from sera from patients. After identifying a candidate protein for analysis, this system allows hundreds of biological samples to be analyzed quantitatively using a single array membrane.

Materials and Methods

Antibodies were purchased from BD PharMingen (San Diego, Calif.). All of cytokines except GROα and granulocyte colony stimulating factor (G-SCF) were obtained from Peptotech (Rochy Hill, N.J.). GROα and G-SCF were the products of BD PharMingen. HPR-conjugated streptavidin was also purchased from BD PharMingen.

Preparation of array membranes. An array of 504 spots (28×18) on a 6×8 cm membrane was generated using a template to guide manual spotting onto a membrane. Capture antibodies were diluted with TBS to 100 μg/ml and 0.25 μl of these solutions for protein arrays or 0.5 μl of conditioned media for conditioned media arrays were manually loaded onto membranes in duplicate using a 2 μl pipetman. HRP-conjugated antibody was spotted onto membranes as positive controls and to allow orientation of the arrays. The strips were cut out for experiments.

Array assay of purified cytokines. Membranes with arrays of immobilized capture antibodies were blocked with 5% BSA (Bovine serum albumin)/TBS (0.01 M Tris HCl pH7.6/0.15 M NaCl) for 1 hour. Membranes were then incubated with a single cytokine or various combinations of different cytokines (0.25 μg/ml) prepared in 5% BSA/TBS for 2 hours at room temperature. After extensive washing with TBS/0.1% Tween 20 (3 times, 5 min each) and TBS (2 times, 5 min each) to remove unbound cytokines, membranes were incubated individually or collectively with biotin-conjugated anti-cytokine antibodies (from 2.5 μg/ml to 0.25 μg/ml). Membranes were then washed to remove unbound anti-cytokine antibodies. Membranes were then incubated with HRP-conjugated streptavidin (2.5 pg/ml) for 1 hour at room temperature. Unbound HRP-conjugated streptavidin was then washed out with TBS/0.1% Tween and TBS. Finally, the presence of bound HRP-conjugated streptavidin, and therefore, the presence of bound cytokine, was detected by ECL in accordance to manufacturer's instructions as described previously.

Protein array assay for detection of cytokines from conditioned media and sera. Ten ml of 50 fold diluted conditioned media and one ml of 10 fold-diluted sera were incubated with membrane arrays for detecting cytokines. To prepare conditioned media, human glioblastoma cells U251 (Huang et al., Cancer Res. 58: 5089-5096 (1998); Huang et al., Cancer Res. 55: 5054-5062 (1995)) were plated in 35 mm tissue culture dishes at a density of 4×10⁵ cells per dish. Cells were cultured with Dulbecco's modified Eagle's medium (DMEM) containing 10% fetal calf serum (FCS) for 24 hours. The complete culture medium was then replaced with serum-free DMEM. Cells were then stimulated for 48 hours in the presence or absence of 50 ng/ml of recombinant human TNFα. The supernatants were then collected, centrifuged at 1,000 g, aliquoted and stored at −80° C. until testing. Patient's sera were obtained from Department of Gynecology and Obstetrics, Emory University School of Medicine.

Conditioned medium arrays. Hybond ECL membranes were soaked with anti-MCP-1 capture antibody at a concentration of 20 μg/ml for 4 hours at 4° C. Membranes were then air-dried. 0.5 μl of conditioned media from different sources were spotted onto the membranes. After blocking with 5% BSA/TBS, membranes were incubated with biotin-conjugated anti-MCP-1 antibody. After washing, membranes were incubated with HRP-conjugated streptavidin. The signals were visualized by ECL.

ELISA. Conventional ELISA was performed as per manufacturer's instructions. Essentially 96 well ELISA plates were coated overnight at 4° C. using 50 μl of 8 μg/ml capture antibodies. 1% BSA/PBS was used as a blocking buffer. 100 μl of conditioned media and 2 fold diluted patients' sera and different concentrations of standard cytokines were added to each well in duplicate. Unbound materials were removed by washing with PBS/1% Tween. 100 μl of 1 μg/ml biotinylated anti-cytokine detection antibody were added to each well. The plates were incubated for 1 hour at room temperature. After washing, 100 μl of streptavidin-HRP conjugated antibodies was added to each well and incubation was continued for 30 minutes at room temperature. Extensive washing followed the incubation. Color development to detect binding was achieved by incubation of the assay components with a substrate solution containing ethylbenzthiazoline sulphonate. O.D. at 405 nm was determined by use of a microplate reader. A standard curve was generated using Sigma plot and the concentrations of different samples were determined from the standard curve.

Densitometry. The intensities of signals were scanned and quantitated by densitometry in accordance with the manufacturer's instructions (Bio-Rad, Hercules, Calif.).

Results

Specificity of protein arrays to detect cytokines. To develop a practical protein array system with high specificity and sensitivity, we took the advantage of specificity of ELISA, sensitivity of ECL and high-throughput of microspot. A pair of antibodies, which recognize two different epitopes of the same antigen were used. One antibody is immobilized onto a membrane and functions as a capture protein. The second antibody is labeled with biotin and functions as a detection protein. The binding of the detection protein is then detected by ECL wherein the biotin is used to attach an HRP-conjugated streptavidin and the HRP generates a detectable signal. Previously, we have demonstrated that up to six different cytokines and 8 different antibodies can be simultaneously detected. Here we examined whether this approach can be extended to detect as many as 24 cytokines from cell culture conditioned media and patient's sera.

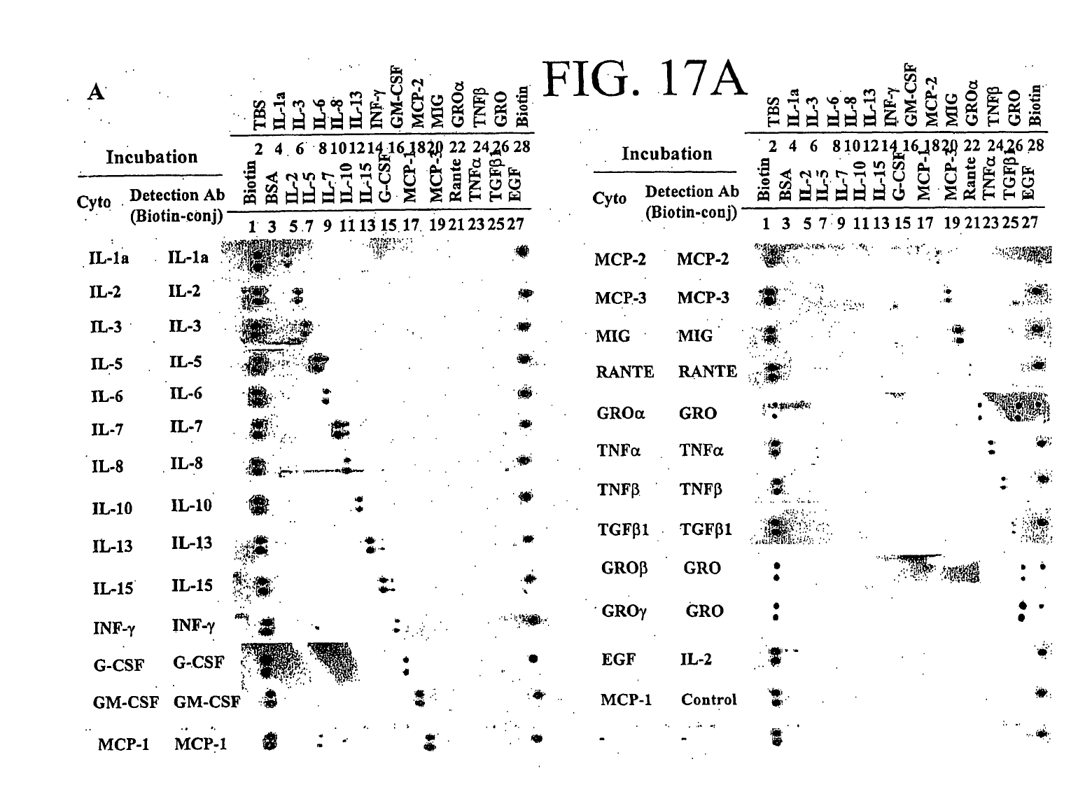

Twenty-four cytokines were assayed using this approach as shown in FIG. 17. Capture antibodies against individual cytokines and other controls were spotted onto Hybond ECL membranes. The membranes were then first incubated with individual cytokines, then incubated with a corresponding biotin-conjugated antibody. As shown in FIG. 17A, specific signals were observed for all twenty-four cytokines. A weak cross-reaction between monocyte chemotactic protein-1 (MCP-1) and interleukin-3 (IL-3) was noticed. Anti-growth-regulated oncogene (GRO) antibody recognizes all three members of GRO: GROα, GROβ and GROγ. When the membrane was incubated with epidermal growth factor (EGF), followed by an irrelevant antibody against IL-2, no signal was detected. When the membrane was incubated with MCP-1 alone without detection antibody (biotin-conjugated anti-MCP-1), no signal was detected. This demonstrates the high level of specificity associated with this assay. The sensitivity and dynamic range of this array were determined by incubation of array membranes with different concentrations of cytokines, such as MCP-1 and IL-2. The intensity of signals were then scanned by Bio-Rad densitometry and plotted against different concentrations of cytokines. The assay can detect the protein level at pg/ml level with the dynamic range spanning about 4 to 5 orders (FIG. 17B).

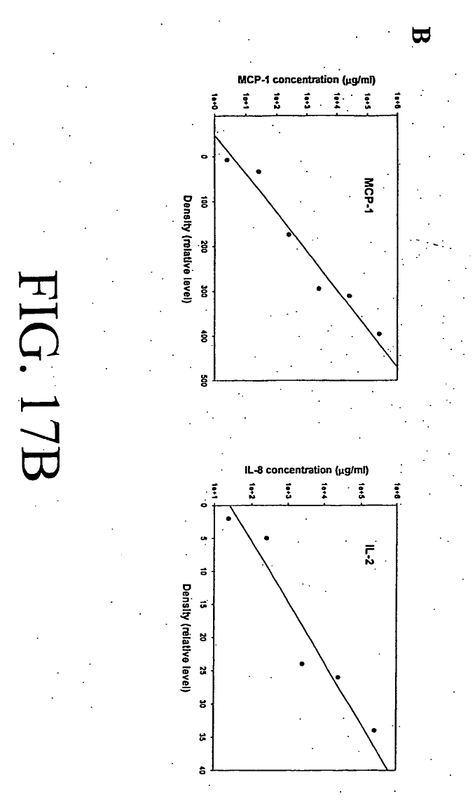

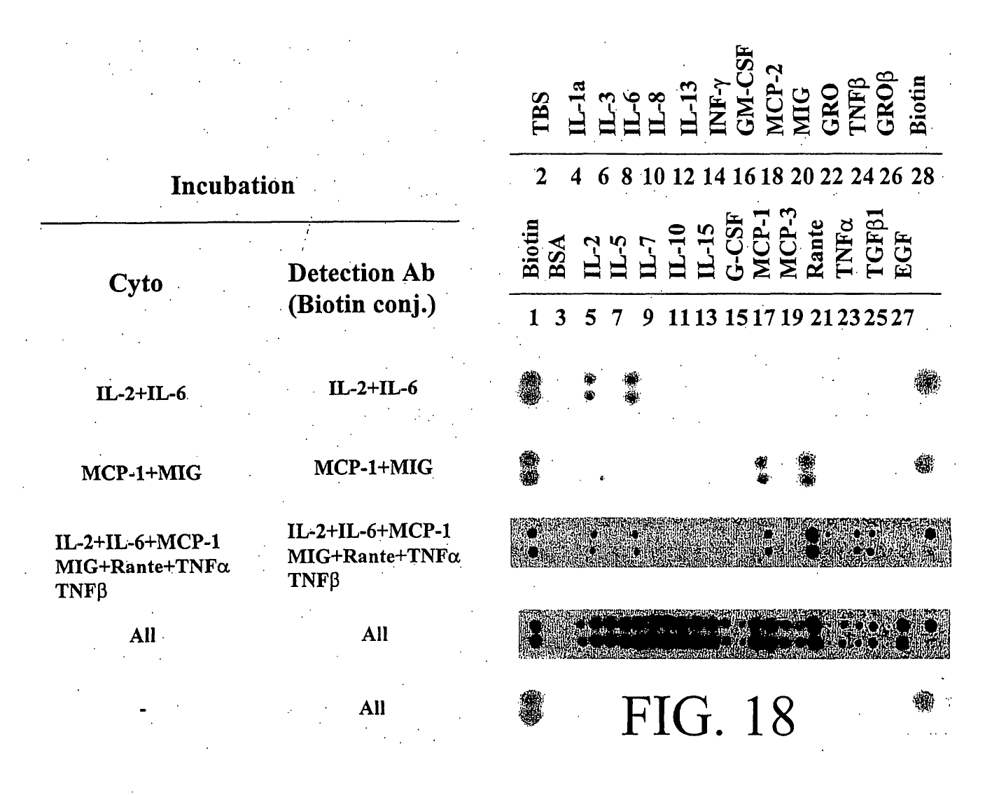

To demonstrate that this is a feasible approach to detect multiple cytokines simultaneously, membranes with different immobilized antibodies were incubated with different combinations of cytokines and corresponding antibodies. Described in FIG. 18, these membrane array assays resulted in the expected patterns of binding and demonstrate the ability to detect specific analytes.

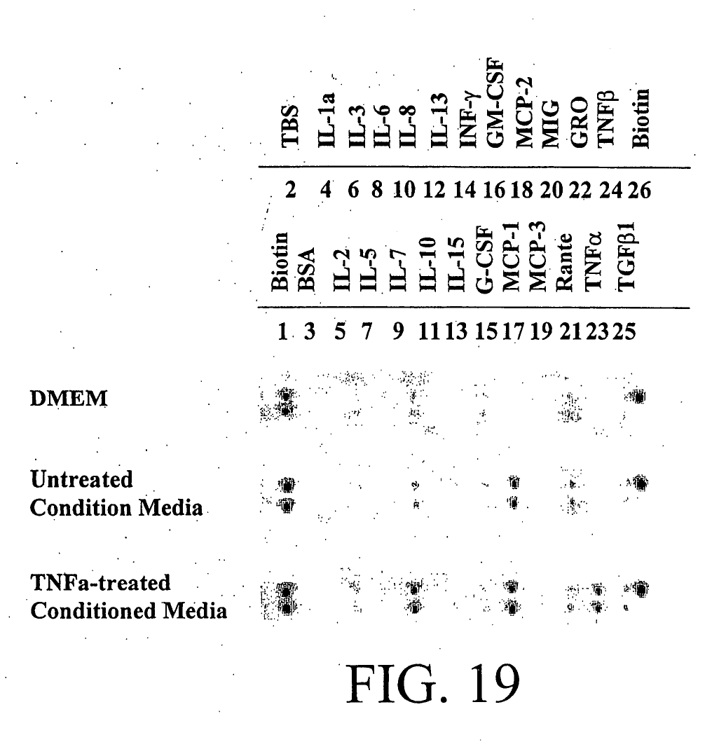

Detection of cytokine expression from conditioned media and patient's sera. A challenging question facing the antibody-based microarray was whether it could be used to directly detect protein expression, i.e., the presence of a particular protein product, in conditioned media, patient's specimen, crude cell lysate or crude tissue lysate. To answer that question, conditioned media collected from different sources were tested for the presence of cytokines. As shown in FIG. 19, several cytokines are differentially expressed in human glioblastoma U251 cells treated with or without tumor necrosis factor α (TNFα). As a control, there was not any significant expression of cytokines detected in medium alone. Densitometry was used to quantify the levels of expression and showed that the MCP-1 level increased 2.5 fold in TNFα-treated U251 cells compared with the untreated cells and IL-8 level increased 17 fold. These results were independently confirmed by ELISA as shown in Table 3. Previous studies demonstrated that TNFα up-regulated MCP-1 expression (Desbaillets et al., Int. J. Cancer, 58: 240-247 (1994)). This is consistent with our protein array data. Furthermore, our result also suggests that TNFα can up-regulate IL-8 expression in human glioblastoma U251 cells, providing new information on the regulation of mediators by TNFα.

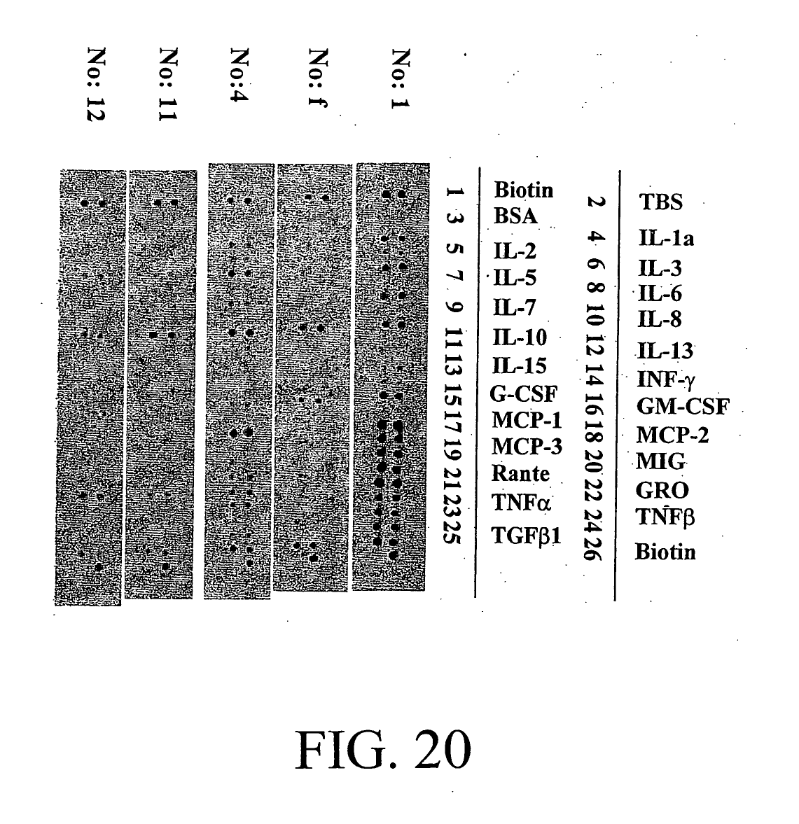

We also tested the detection of cytokines from patient's sera. Several patients' sera were screened for cytokine expression. Cytokine detection arrays were incubated with several patients' sera. As indicated in FIG. 20, specific cytokines were detected in samples from different patients. The relative levels of MCP-1, IL-8 and TNFα expression were determined by densitometry (Table 3) and are in general agreement with results obtained using a conventional ELISA assay.

Conditioned medium microarrays for high throughput molecular profiling. Through use of the protein array techniques outlined here, simultaneous detection of multiple cytokines and identification of the key molecules important in specific cases or pathologies can be achieved. To further investigate the role of key molecules in a given pathology or situation, it is generally necessary to examine their expression in many samples. As previously described, examination of their expression in many samples is costly and time-consuming process. At this stage of investigation, limited numbers of protein molecules will be examined. Therefore, it will be much efficient to screen as many samples as possible at one time. One of the difficulties in performing this type of experiment is that the sensitivity is not high enough to allow sample volumes to be significantly scaled down. Another potential problem is related to the variation of antigens. For example, different antigens often have considerably different abilities to bind to membranes.

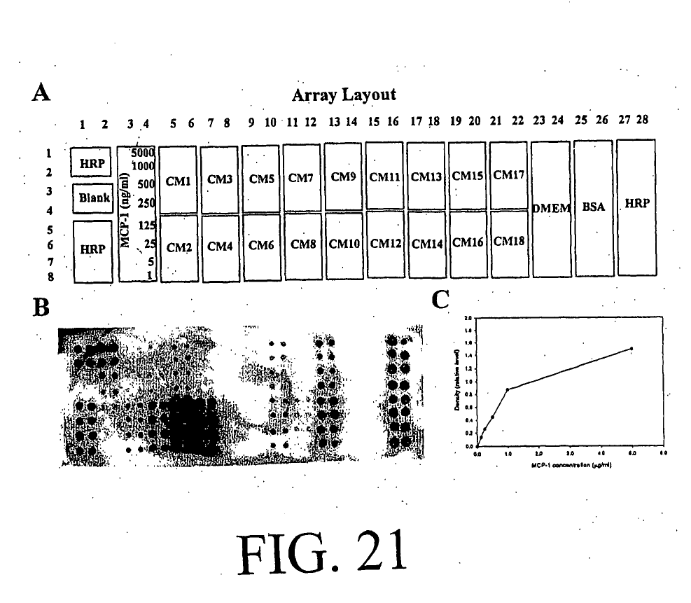

To overcome these two major obstacles, we coated membranes homogenously with an antibody specific for a corresponding antigen. For example, to detect MCP-1 levels in different samples, membranes were coated with capture anti-MCP antibody. Different samples were then spotted onto the membranes. The expression levels of MCP-1 were then detected by biotin-conjugated anti-MCP-1 antibody coupled with ECL system. We found that coating of the membrane with a specific antibody increased detection sensitivity at least 100 fold. Then we tested the potential of this array system. 324 loci in a single membrane pre-coated with anti-MCP-1 capture antibody were spotted with conditioned media from different sources (FIG. 21A). Membrane was then incubated with biotin conjugated anti-MCP antibody. The results in FIG. 21B indicated that this is a feasible approach to simultaneously detect cytokine levels from many conditioned media. Since the MCP-1 standards are spotted on the same membrane and are measured on the same membrane, the concentrations of MCP-1 in samples can be determined directly with precision and accuracy by comparison to the standard curve (FIG. 21C).

### EXAMPLE 4

### Profiling of Human Cytokines in a Variety of Biological Processes by Protein Arrays

This example demonstrates the manufacture and use of a higher density array system to simultaneously detect 35 cytokines. By applying this system, we demonstrated that monocyte chemoattatic protein-1 may be implicated in tumor suppression by gap junction protein, connexin 43; interleukin 8 levels are associated with p53 status in breast cancer cells and the levels of monocyte chemoattactic protein-1 were significantly decreased in patient in response to vitamin E supplementation. In addition, our system can be easily adapted to chip platform. Multiple cytokine expression from different sources can be detected using protein chips.

Previously, we have developed a novel ELISA-based protein array system in which up to 23 cytokines can be simultaneously detected from physiopathological samples with high sensitivity and specificity (Huang, “Detection of multiple proteins in an antibody-based protein microarray system,” J Immunol Methods 255:1-13 (2001); Huang et al., “Simultaneous detection of multiple cytokines from conditioned media and patient's sera by an antibody-based protein array system,” Anal Biochem 294:55-62 (2001); Huang, “Simultaneous detection of multiple proteins with an array-based enzyme-linked immunosorbent assay (ELISA) and enhanced chemiluminescence (ECL),” Clin Chem Lab Med 39:209-214 (2001)). Here, we have expanded our detection up to 35 cytokines simultaneously and have applied this technology to resolve some real biological questions. Thus, we have demonstrated that human cytokine arrays could be used to study molecular mechanisms of tumor suppression, profile human cytokine expression from tumor cells and monitor human cytokine expression in response to vitamin E. In addition, the technology we developed can be easily applied to a protein chip platform. To our knowledge, this is the first demonstration that human cytokine arrays have practical applications in both basic and clinical research.

Results and Discussion

Analysis of Human Cytokine Expression in Cx43-Transfected Cells.

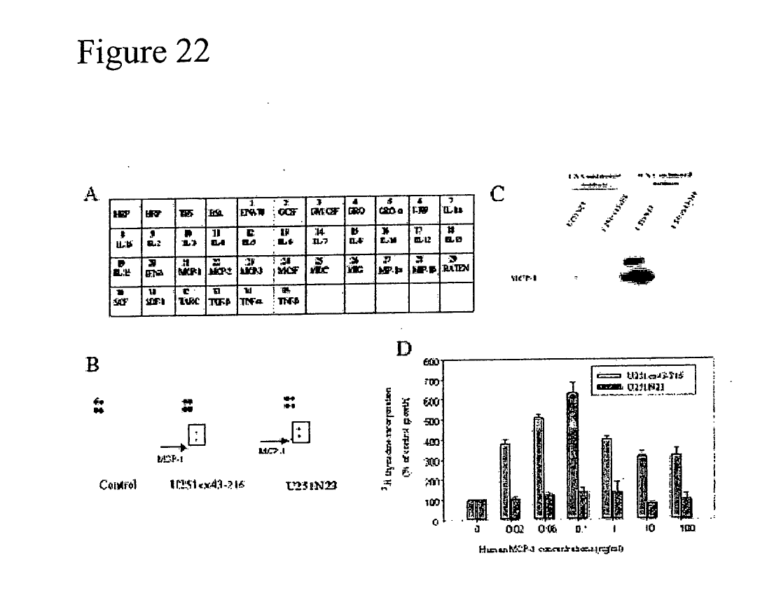

The ability to detect expression of multiple cytokines allowed by the new technology prompted us to apply this technology to resolve some real biological questions. First, the human cytokine array systems developed here was used to address questions related to the molecular mechanisms of tumor suppression. We previously showed that transfection of Cx43 can reverse the transformed phenotypes of human glioblastoma cells (Huang et al., “Reversion of the neoplastic phenotype of human glioblastoma cells by connexin 43 (cx43),” Cancer Res 58:5089-5096 (1998); Huang et al., “Connexin 43 (cx43) enhances chemotherapy-induced apoptosis in human glioblastoma cells,” Int J Cancer 92:130-138 (2001)). To explore the possible molecular mechanisms responsible for this tumor suppression, we screened for potential Cx-43-regulated cytokines in Cx43-transfected and control-transfected cells. As shown in FIG. 22 B, expression of MCP-1 was significantly reduced in Cx43-transfected cells. All other cytokines are similar between Cx43-transfected and control-transfected cells. To further confirm the human cytokine array results, we performed immunoprecipitation of conditioned media from Cx43-transfected cells and control-transfected cells with antibody against MCP-1. The immunoprecipitated complex was then separated by SDS PAGE and the levels of MCP-1 protein were detected by Western Blot using antibody against MCP-1. As shown in FIG. 22 C, the MCP-1 protein was predominantly detected in conditioned media from control-transfected cells (U251N23), but not from Cx43-transfected cells (U251cx43-216).

The results suggest that down-regulation of MCP-1 in Cx43-transfected cells may contribute to the reversion of transformed growth by Cx43. To test this possibility, recombinant MCP-1 was added to the tissue culture media of Cx43-transfected cells (U251cx43-216) and control-transfected cells (U251N23) and cell proliferation rates were determined by ³H thymidine incorporation assay. As shown in FIG. 22 D, addition of MCP-1 specifically stimulated cell proliferation rates in Cx43-transfected cells but not in control-transfected cells, suggesting the involvement of MCP-1 in Cx43-mediated growth control. In contrast, neutralization antibody against MCP-1 inhibited cell proliferation rates of control-transfected cells but not Cx43-transfected cells.

Profiling of Human Cytokine Expression in a Variety of Human Breast Cancer Cells.

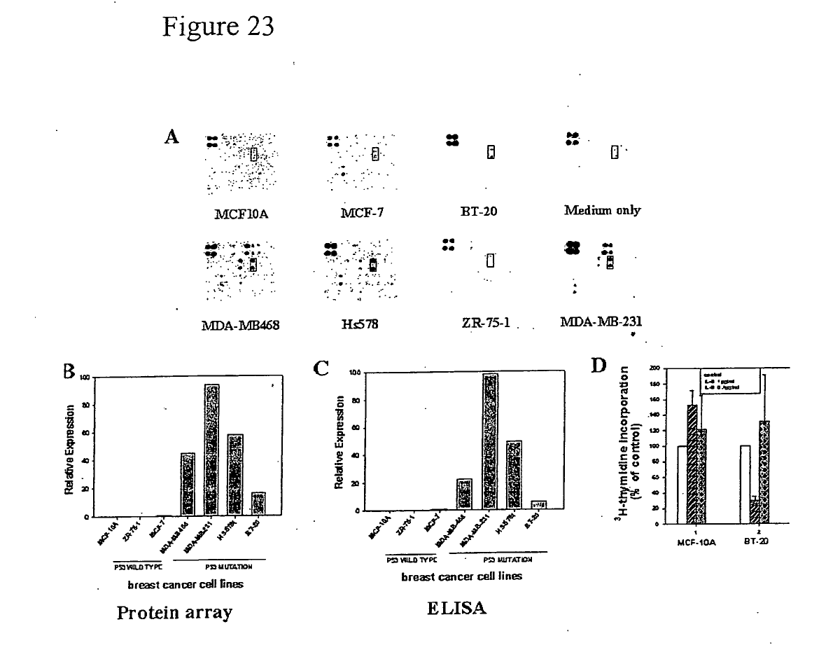

Then we applied the system to analyze the profiling of human cytokines in human breast cancer cell lines. Conditioned media from different human breast cancer cell lines were assayed for their cytokine profiling by using cytokine array system described above. Several representative results were shown in FIG. 23 A. Several cytokines were differentially secreted from a number of human breast cancer cell lines. As a control, no significant levels of cytokines were detected from cell-free medium alone. The relative expression levels of cytokines were determined by densitometry. When the cytokine levels and p53 status was compared, we found that p53 status was related to the IL-8 levels. In the cells containing wild type p53, very low amount of IL-8 was detected, whereas, in the cells harboring mutant type p53, significantly high levels of IL-8 were noticed (FIG. 23 B). The expression levels were confirmed by ELISA (FIG. 23 C). To determine the biological significance of IL-8 in human breast cancer cells, cell proliferation assays were carried out in IL-8 low expressing cells (MCF-10A) and IL-8 highly expressing cells (BT-20). As shown in FIG. 23 D, addition of antibodies against IL-8 specifically decreased cell proliferation rates in human breast cancer cells expressing high levels of IL-8. Our results suggest that a new p53-IL-8 pathway is important in the regulation of human breast cancer cell growth. Further characterization of this pathway may enhance our understanding of the development of human breast cancer and provide a new target for clinical intervention.

Monitoring of Human Cytokine Expression in Response to Vitamin E.

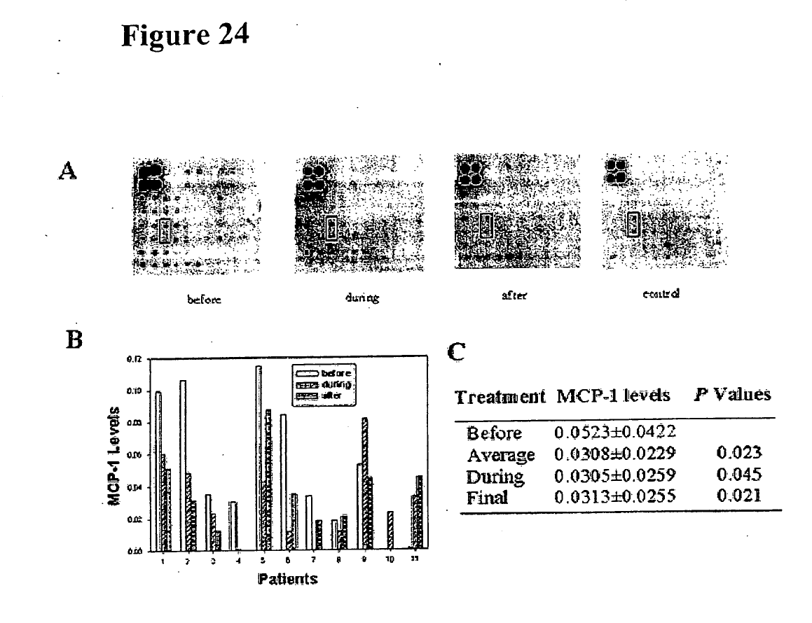

The human cytokine array system can also be used to identify possible targets of chemoprevention. To illustrate this, we analyzed cytokine levels from 11 patient's serum before and after vitamin E supplementation. We have previously shown that vitamin E supplementation increased vitamin E levels in patient's sera (Santanam et al., “Vitamin E supplementation decreases autoantibodies to oxidized lipid-protein complexes,” J. Medicinal Food 1:247-251 (1998)). Representative results were shown in FIG. 24 A. Generally, supplementation of vitamin E decreased a number of cytokine expression. The relative levels of MCP-1 were then determined by densitometry as shown in FIG. 24 B and the difference was statistically analyzed (FIG. 24 C). We found that MCP-1 levels were significantly down-regulated after supplementation with vitamin E, suggesting that MCP-1 may be a molecular target of antioxidant supplementation. This observation represents the first report of a potential molecular target of vitamin E in vivo. Further studies may reveal valuable information about molecular mechanisms of vitamin E supplementation in the chemoprevention and identify novel targets.

Detection of Cytokine Levels Using Protein Chips.

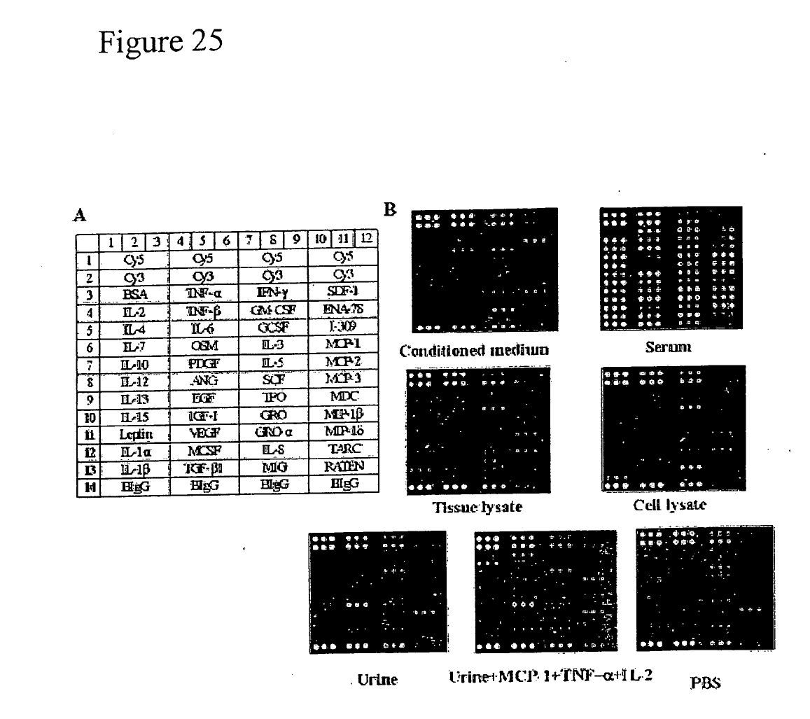

Recently, several labs and companies have produced protein chips with varying levels of success. To test whether our system can be successfully adapted to a chip platform, we examined the cytokine expression from different biological samples: conditioned medium, patient's serum, cell lysate, tissue lysate and urine using Hydrogel chips. In this case, 43 capture antibodies were printed onto Hydrogel chips to generate human cytokine chips. Our data showed that there is no cross-reactivity among those 43 cytokines in the array assay. The cytokine chips were then incubated with 50 μl of different biological samples, followed by a incubation with a cocktail containing 43 biotinylated detection antibodies and cy3-conjugated streptavidin. As shown in FIG. 25, specific cytokines could be detected in different samples in the cytokine chips, suggesting that our system is readily adapted to protein chip format and such protein chips can be used to detect cytokines from different biological sources. In general, the results were consistent with that obtained from membrane-based arrays and ELISA.

In summary, the system we describe in this example has several important features. The system can be used to detect multiple human cytokines from a variety of biological sources such as, but not limited to, conditioned medium, serum, cell lysate, tissue lysate and urine. Our system can be both a membrane-based system and a chip-based system. The membrane format allows designing arrays in a simple, cheap and flexible way. Among the particular advantages to the membrane-based system are that no sophisticated equipment is required in the entire process. Consequently, it is adaptable for use by nearly all of the research community. The protein chip format also has its own particular advantages. Among these are its ability to be used in a high-throughput approach using methods and compositions that are in widespread use in the field. Variation from one assay to the next is quite acceptable for almost all purposes and is usually within the range of approximately 10%. This sensitivity is high enough to detect the change of most biological processes.

As with any discovery method, the data obtained by human cytokine arrays may not be sufficient to explain the molecular mechanisms of biological processes. However, our data do uncover new molecular targets in those processes and suggest hypothesis that can subsequently tested by traditional molecular and cell biology methods or followed by further scientific exploration. This system holds a great promise in basic and clinical research.

Materials and Methods

Materials

All pair antibodies were purchased either from BD PharMingen (San Diego, Calif.) or from R&D (mineapolis, Minn.). Cytokines were obtained from Propetech (Rochy Hill, N.J.), BD PharMingen and R&D. HPR-conjugated streptavidin was purchased from BD PharMingen. Cy3-conjugated streptavidin was the product of Rockland (Gilbertsville, Pa.).

Preparation of Array Membranes

The preparation of array membranes was essentially as described elsewhere in this application and in the references incorporated herein by reference. A computed generated-template was used to guide to spot solution onto membranes. 0.20 μl of capture antibodies (200 μg/ml) were manually loaded onto membranes by a 2 μl pipetman in duplicate. HRP-conjugated antibody was spotted onto membranes as positive control and identification of orientation of arrays.

Array Assay of Human Cytokine Expression.

Membranes that included immobilized capture antibodies were blocked with 5% BSA (Bovine serum albumin)/TBS (0.01 M Tris HCl pH7.6/0.15 M NaCl) for 1 hour. Membranes were then incubated with 1 ml of a single or a combination of different cytokines (100 ng/ml) or 1 ml of conditioned media or 1 ml of 10 fold diluted patient's sera prepared in 5% BSA/TBS for 2 hours at room temperature. After an extensive wash with TBS/0.1% Tween 20 (3 times, 5 min each) and TBS (2 times, 5 min each) to remove unbounded cytokines, membranes were incubated individually or collectively with biotin-conjugated anti-cytokine antibodies. Further washes followed and then the membranes were incubated with HRP conjugated streptavidin (2.5 pg/ml) for 1 hour at room temperature. Unbound materials were washed out with TBS/0.1% Tween and TBS. Finally the signals were detected by ECL system.

ELISA

Conventional ELISA was performed according to the manufacturer's instructions. Essentially 96 well ELISA plates were coated overnight at 4° C. using 50 μl of 8 μg/ml capture antibodies. 1% BSA/PBS were used as a blocking buffer. 100 μl of conditioned media and different concentrations of standard cytokines were added to each well in duplicate. Unbound materials were washed out with PBS/1% Tween. 100 μl of 1 μg/ml biotinylated anti-cytokine detection antibody were added to each well. The plates were incubated for 1 hour at room temperature. After washing, 100 μl of streptavidin-HRP conjugated antibodies were added to well and incubation was continued for 30 minutes at room temperatures. Further extensive washes were then used. Color development was done by incubating with the calorimetric substrate solution containing ethylbenzthiazoline sulphonate. The development of color was monitored by determination of the O.D. at 405 nm by use of a microplate reader.

Densitometry

The intensities of signals were scanned and quantitated by densitometry (Bio-Rad, Hercules, Calif.).

³H Thymidine Incorporation Assays.

The experiment was performed as described in Huang et. al (Huang et al., “Egr-1 negatively regulates human tumor cell growth via the DNA-binding domain,” Cancer Res 55:5054-5062 (1995); Huang et al., “A biological role for Egr-1 in cell survival following ultra-violet irradiation,” Oncogene 10: 467-475 (1995)). Cells were seeded in 96-well plates. 24 hr later, cells were incubated in the presence of cytokine or antibody for 48 hours. 0.5 μCi of ³H thymidine was added to each well and incubation was continuous for 24 hr. The incorporated ³H thymidine was then determined by a scintillation counter.

Human Cytokine Chip Technology.

300 pL of capture antibodies (500 μg/ml) were printed onto Hydrogel chips (Packard Bioscience, Meriden, Conn.) using the Biochip Arrayer (Packard Bioscience). After blocking, the chips were incubated with 50 μl of different samples at room temperature for 2 hr. The chips were then washed to remove unbound components. Biotin-labeled detection antibody cocktail was added (50 μl/chip) and incubated at room temperature for 1 hr. After the washing, cy3 labeled streptavidin was added and the chips were incubated at room temperature for 1 hr. The excess amount of cy3 streptavidin was removed and the resulting signals were scanned by laser scanner (Affymetrix, Santa Clara, Calif.). A series of diluted Cy3 streptavidin, cy5 streptavidin and Biotin IgG (BIgG) were included as a positive control. BSA was used as negative control.

### REFERENCES

The following references are incorporated herein by reference for their teachings related the production and use of arrays generally, but more particularly for the purpose of illustrating the knowledge of one of skill in the art at the time of, or at a time subsequent to, the publication of the listed references. This knowledge includes; the fabrication of membrane and chip-based arrays; the application of capture agents to the membranes or chip substrates to form arrays; the attachment of capture agents to the array; the detection methods used for detecting the presence of an analyte or protein of interest; the quantification of an analyte or protein of interest; the analysis of data derived from the use of the arrays; and for the preparation of samples for use with the presently disclosed arrays and methods.
1. Fields S: Proteomics. Proteomics in genomeland. Science 2001, 291:1221-1224.
2. Mills J C, Roth K A, Cagan R L, Gordon J I: DNA microarrays and beyond: completing the journey from tissue to cell. Nat Cell Biol 2001, 3:E175-E178.
3. Schulze A, Downward J: Navigating gene expression using microarrays—a technology review. Nat Cell Biol 2001, 3:E190-E195.
4. Dutt M J, Lee K H: Proteomic analysis. Curr Opin Biotechnol 2000, 11:176-179.
5. MacBeath G, Schreiber S L: Printing proteins as microarrays for high-throughput function determination [see comments]. Science 2000, 289 :1760-1763.
6. de Wildt R M, Mundy C R, Gorick B D, Tomlinson I M: Antibody arrays for high-throughput screening of antibody-antigen interactions. Nat Biotechnol 2000, 18:989-994.
7. Mendoza L G, McQuary P, Mongan A, Gangadharan R, Brignac S, Eggers M: High-throughput microarray-based enzyme-linked immunosorbent assay (ELISA). Biotechniques 1999, 27:778-6, 788.
8. Haab B B, Dunham M J, Brown P O: Protein microarrays for highly parallel detection and quantitation of specific proteins and antibodies in complex solutions. Genome Biol 2001, 2:RESEARCH0004-
9. Moody M D, Van Arsdell S W, Murphy K P, Orencole S F, Burns C: Array-based ELISAs for high-throughput analysis of human cytokines. Biotechniques 2001, 31:186-4.
10. Bussow K, Cahill D, Nietfeld W, Bancroft D, Scherzinger E, Lehrach H, Walter G: A method for global protein expression and antibody screening on high-density filters of an arrayed cDNA library. Nucleic Acids Res 1998, 26:5007-5008.
11. Bussow K, Nordhoff E, Lubbert C, Lehrach H, Walter G: A human cDNA library for high-throughput protein expression screening. Genomics 2000, 65:1-8.
12. Lueking A, Horn M, Eickhoff H, Bussow K, Lehrach H, Walter G: Protein microarrays for gene expression and antibody screening. Anal Biochem 1999, 270:103-111.
13. Ge H: UPA, a universal protein array system for quantitative detection of protein-protein, protein-DNA, protein-RNA and protein-ligand interactions. Nucleic Acids Res 2000, 28:e3-
14. Zhu H, Klemic J F, Chang S, Bertone P, Casamayor A, Klemic K G, Smith D, Gerstein M, Reed M A, Snyder M: Analysis of yeast protein kinases using protein chips. Nat Genet 2000, 26:283-289.
15. Zhu H, Bilgin M, Bangham R, Hall D, Casamayor A, Bertone P, Lan N, Jansen R, Bidlingmaier S, Houfek T et al: Global analysis of protein activities using proteome chips. Science 2001, 293:2101-2105.
16. Joos T O, Schrenk M, Hopfl P, Kroger K, Chowdhury U, Stoll D, Schorner D, Durr M, Herick K, Rupp S et al: A microarray enzyme-linked immunosorbent assay for autoimmune diagnostics. Electrophoresis 2000, 21:2641-2650.
17. Huang R P: Detection of multiple proteins in an antibody-based protein microarray system. J Immunol Methods 2001, 255:1-13.
18. Huang R P, Huang R, Fan Y, Lin Y: Simultaneous detection of multiple cytokines from conditioned media and patient's sera by an antibody-based protein array system. Anal Biochem 2001, 294:55-62.
19. Huang R P: Simultaneous detection of multiple proteins with an array-based enzyme-linked immunosorbent assay (ELISA) and enhanced chemiluminescence (ECL). Clin Chem Lab Med 2001, 39:209-214.

### EXAMPLE 5

### Profiling of Protein Expression by Biotin-Labeled and Antibody-Based Protein Arrays

In this example, we describe a biotin-labeled and antibody-based protein array system to simultaneously detect multiple cytokines from biological samples.

Results

In this new approach, proteins from a variety of biological sources are labeled with biotin. The biotin-labeled proteins are then incubated with arrays having antibodies as capture proteins. The particular support used here is a chip, but the method is equally applicable to the use of membrane-based arrays. In the example, targeted proteins were captured by the array antibodies spotted on protein chips. The presence of targeted proteins was detected using cy3- or cy5-conjugated streptavidin and the signals were imaged by laser scanner. Most proteins at concentration of pg/ml can be detected with a detection limit at low to mid fg levels. The useful dynamic detection range of this system is at least about 4 to 5 orders of magnitude. The system also can be easily adapted to two-color binding assay, allowing measurement of levels of proteins in a test sample with respect to a reference sample at the same chip. To demonstrate the potential application of this technology, we applied this technology to profile human cytokines, chemokines, growth factors, angiogenic factors and proteases in a variety of human breast cancer cell lines and identify the key molecular targets from monocytes upon activation by lipopolysaccharide (LPS). These results suggest that biotin-labeled and antibody-based chip technology can provide a practical and powerful means to profile hundreds and thousands of protein in research and clinical applications.

Materials and Methods

Materials

All pair antibodies were purchased either from BD PharMingen (San Diego, Calif.) or from R&D (Minneapolis, Minn.). Cytokines were obtained from Propetech (Rochy Hill, N.J.), BD PharMingen and R&D. HRP-conjugated streptavidin was purchased from BD PharMingen. Cy3-conjugated streptavidin was the product of Rockland (Gilbertsville, Pa.).

Human Cytokine Chip Technology.

300 pL of capture antibodies (500 μg/ml) were printed onto Hydrogel chips (Packard Bioscience, Meriden, Conn.) using the Biochip Arrayer (Packard Bioscience). After blocking, the chips were incubated with 50 μl of different samples at room temperature for 2 hr. The chips were then washed to remove unbound components. Biotin-labeled detection antibody cocktail was added (50 μl/chip) and incubated at room temperature for 1 hr. After wash, cy3- or cy5-labeled streptavidin was added and the chips were incubated at room temperature for 1 hr. The excess amount of streptavidin was removed and the signals were scanned by laser scanner (Packard, Meriden, Conn.). A series of diluted Cy3 streptavidin, cy5 streptavidin and Biotin IgG (BIgG) were included as positive control. BSA was used as negative control.

Results

In developing systems for the detection of multiple proteins, it can be preferred to minimize the number of detection proteins. One approach to achieve this goal is to develop detection antibody which can recognize a common domain such that only a few detection antibodies is required for the entire array system. A second approach is to label proteins to be detected with fluorescent dye, gold particles or biotin, so that the use of a separate detection protein is not required. The second approach is demonstrated in this example.

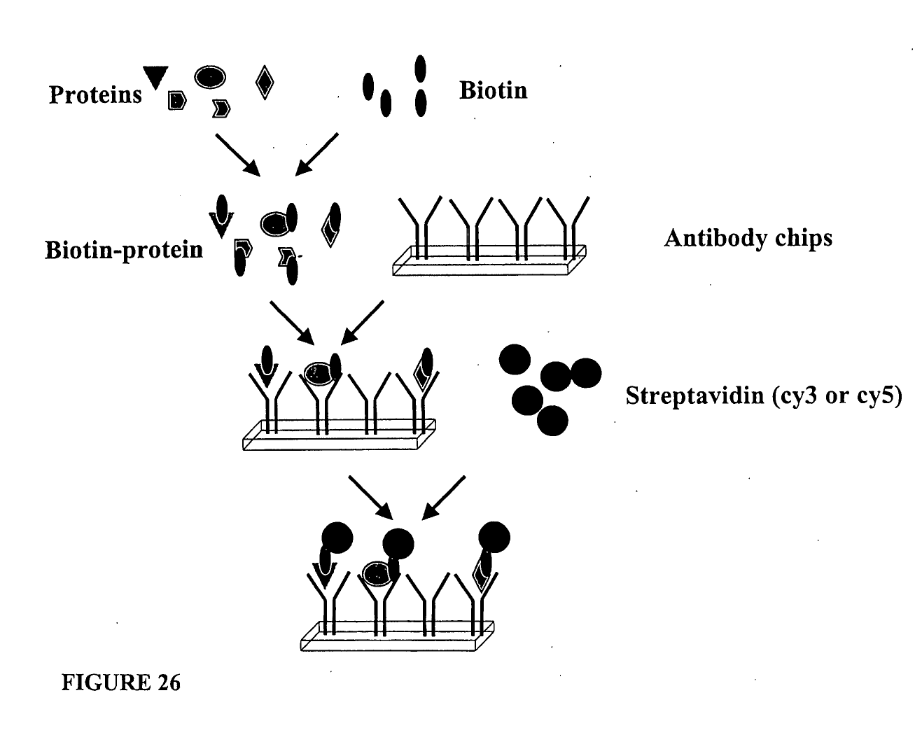

The proteins to be detected/identified were labeled with biotin. The biotinylated proteins were then incubated with array membranes or chips having specific, known sets or subsets of capture proteins. After extensive washes to remove any unbound proteins, the array membranes or chips were incubated with streptavidin conjugated with cy3 or cy5 or HRP as shown in FIG. 26.

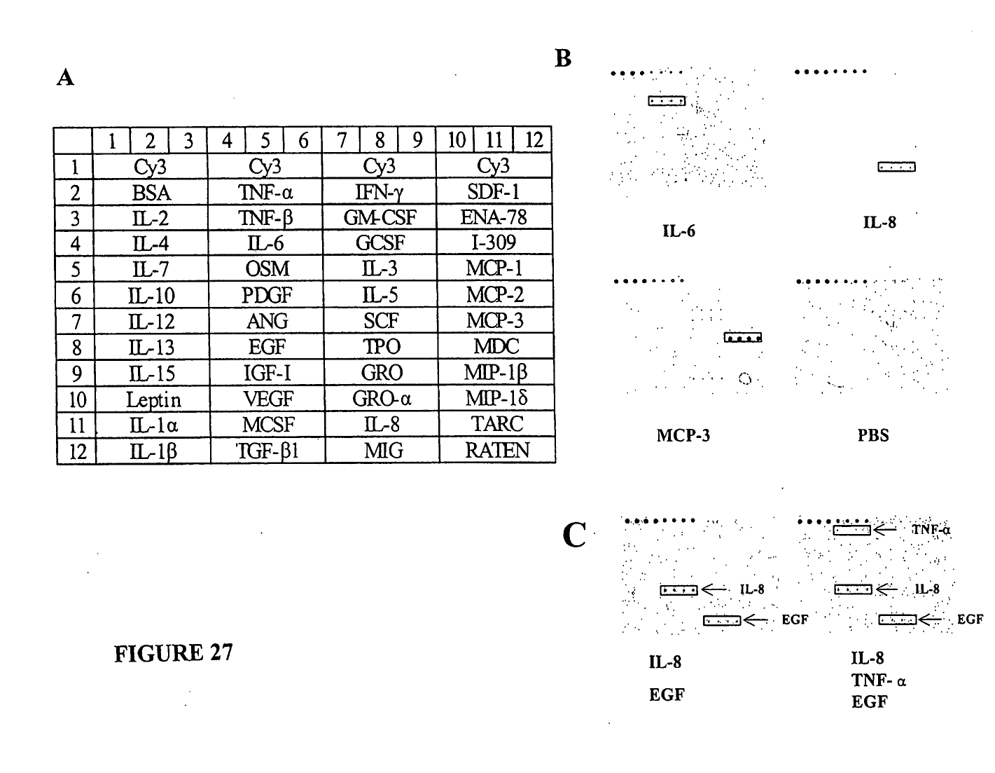

As a first step, we tested the sensitivity and specificity of the assay. Individual recombinant cytokine was labeled with biotin using Pierce biotin-label kit. Free biotin was then removed by dialysis. Human cytokine chips were incubated with biotin-labeled cytokine. Excess, unbound cytokine was then removed by an extensive wash. The chips were then incubated with cy3-streptavidin. After washing to remove any unbound cy3-streptavidin, signals were visualized using a laser scanner. Spots of detectable signal corresponding to IL-8, IL-6 and MCP-3 were detected with high specificity (FIG. 27). The specific signals were also detected with a mixture of different biotin-labeled cytokines as shown in FIG. 27. No signal was detected when biotin-labeled solvent was used. These results demonstrated the specificity of our system.

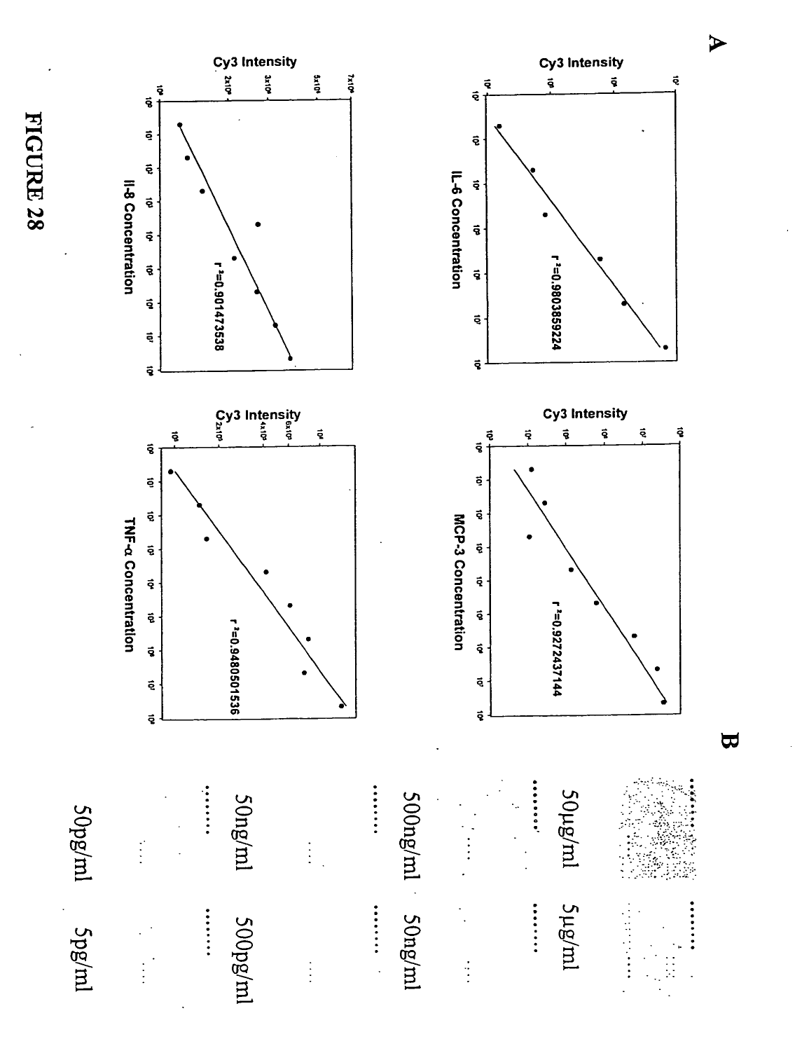

The detection sensitivity was determined by incubation of human cytokine array with different concentrations of biotin-labeled cytokines. As shown in FIG. 28, the detection sensitivity ranged from 0.5 μg/ml to 500 μg/ml. Since 50 μl of biotin-labeled cytokine was used in one experiment, this translated into the detection amount (0.5 μg/ml/20=25 fg). The detection sensitivity of individual cytokines, in this example, is different and is, as one would expect, dependent on the binding affinity between antigen and antibody. Nevertheless, a linear increase in spot intensity was observed with increasing amounts of cytokine in several cytokines we tested.

The variability between measurements to detect the same species of sample on the same membrane (intra-variability) was determined by comparing the signals from 16 duplicate different spots in the same array membrane (Table 4). The variability of spots from different membranes (inter-variability) was determined from 4 different arrays (Table 4). The CV (standard deviation divided by average) was usually less than 10%, suggesting the reproducibility and precision of the system.

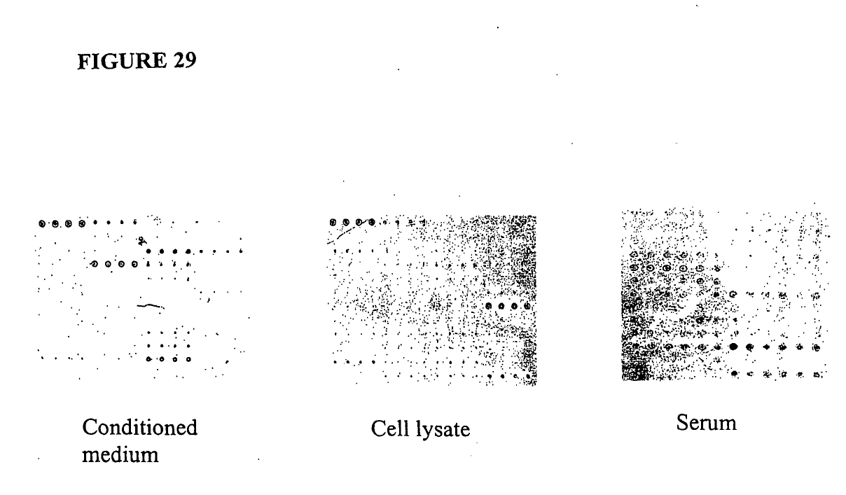

After demonstrating the specificity, feasibility, sensitivity and accuracy of this array system, we tested whether this system, including the use of biotin labeling of analyte protein, can be used to detect the protein expression in real biological samples. Conditioned media, plasma and cell lysate were labeled with biotin. The biotin-labeled samples were then incubated with cytokine chips. As shown in FIG. 29, specific cytokines were detected in samples from different sources, suggesting that the system is practical means to measure the alteration of cytokines at physiological levels. The relative expression levels of MCP-1, IL-8 and TNFα were determined by densitometry (Table 4) and agreed well with results obtained using ELISA.

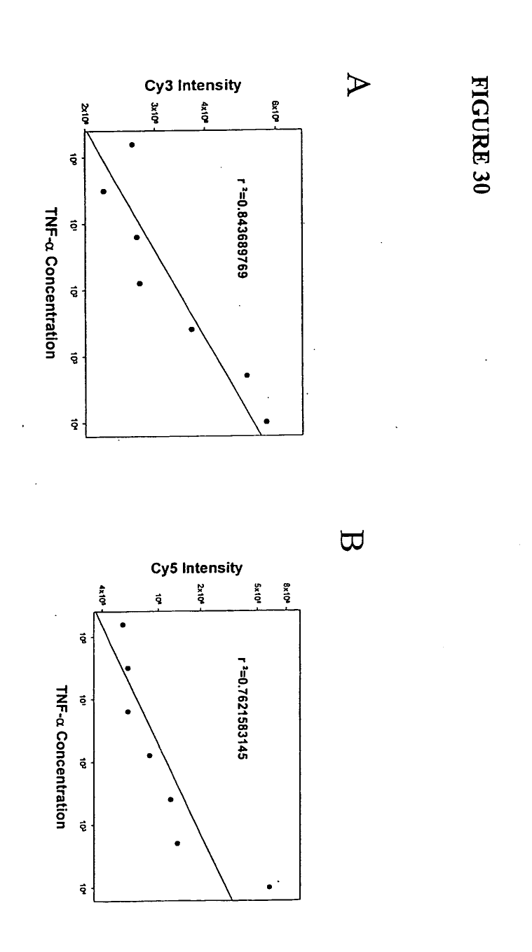

Antibody microarrays can also be used to measure the levels of protein in a test sample with respect to a reference sample, e.g., cancer versus normal cells, stimulated cells versus control, treated versus untreated cells and so on. To demonstrate this, we tested a two-color binding assay, similar to that commonly used with DNA microarrays. First we tested the inflorence of cy3 on the cy5 and vice versa. Fixed amounts of biotin-labeled TNFα incubated with cy3-streptavidin were mixed together with increasing amounts of biotin-labeled TNFα incubated with cy5-streptavidin. Similarly, fixed amounts of biotin-labeled TNFα incubated with cy5-streptavidin were mixed together with increasing amounts of biotin-labeled TNFα incubated with cy3-streptavidin. The mixtures of both cy3 and cy5 samples were then incubated with human cytokine chips. As shown in FIG. 30, linear response curves were obtained with increasing amounts of TNFα as measured by the resulting signal intensities. This result indicates that two samples can be probed using two distinct fluorescent dyes. It also indicates that the system can be used in situations where the two systems compete in the same binding assay. Among the advantages of this two-color approach is that variability attributable to spot size, amount of antibody deposited, and incubation conditions can be controlled for by use of a second signal. By use of such controls, comparisons of the assays and the results of the assays can be made to be more valid. These comparisons can be within a microarray or between microarrays.

One biotin-labeled conditioned medium was incubated with cy3-streptavidin. Another biotin-labeled conditioned medium was incubated with cy5-streptavidin. Both solutions were then incubated with the antibody array that had been prepared on the protein chips. The protein chips were then scanned using a laser scanner. The cy3/cy5 and cy5/cy3 ratios of spot intensities were determined. Just as in the case of the one-color approach, the two-color approach showed similar, and equally useful, results.

Unless defined otherwise, all technical and scientific terms used herein have the same meanings as commonly understood by one of skill in the art to which the disclosed invention belongs. Although any methods and materials similar or equivalent to those described herein can be used in the practice or testing of the present invention, the preferred methods, devices, and materials are as described. Publications cited herein and the material for which they are cited are specifically incorporated by reference. Nothing herein is to be construed as an admission that the invention is not entitled to antedate such disclosure by virtue of prior invention.

Those skilled in the art will recognize, or be able to ascertain using no more than routine experimentation, many equivalents to the specific embodiments of the invention described herein. Such equivalents are intended to be encompassed by the following claims.

TABLE 1
Array Antigens and Corresponding Antibodies

| Antigens | Sources | Cat. No. | Antibodies | Sources | Cat. No |
|---|---|---|---|---|---|
| Bovine IgG | Sigma | I 5506 | Anti-bovine IgG | JIRL | 101-035-003 |
| Chicken IgG | Sigma | I 4881 | Anti-chichen IgG | JIRL | 703-035-155 |
| Goat IgG | Sigma | I 5256 | Anti-goat IgG | SCB | Sc-2033 |
| Guinea pig IgG | Sigma | I 4756 | Anti-guinea pig IgG | JIRL | 706-035-148 |
| Human IgG | Sigma | I 2511 | Anti-human IgG | JIRL | 309-035-082 |
| Mouse IgG | Sigma | I 5381 | Anti-mouse IgG | Sigma | A-4416 |
| Rabbit IgG | Sigma | I 5006 | Anti-rabbit IgG | Sigma | A-6154 |
| Rat IgG | Sigma | I 4131 | Anti-rat IgG | Amersham | NA932 |
| Sheep IgG | Sigma | I 5131 | Anti-sheep IgG | JIRL | 12-342 |
| BSA | Roche | 100 350 | | | |

JIRL: Jackson ImmunoResearch Laboratories;
SCB: Santa Cruz Biotechnology.

TABLE 2
Membranes for Protein Arrays

| | | | | Detection | | Detection | |
|---|---|---|---|---|---|---|---|
| | | | | Of IgGs | | Of cytokines | |
| Membrane | Manufacturer | Cat. No. | Absorption | Bkg | Sensitivity | Bkg | Sensitivity |
| Biotrans | ICN | BNRQ3R | Excellent | ++ | +++ | +++++ | ? |
| Zeta-probe | Bio-Rad | 162-0155 | Good | ++ | +++ | | |
| Colony/plaque | NEN | NEF-978X | Very Good | ++ | +++ | | |
| screen | | | | | | | |
| Hybond-N+ | Amersham | PRN303B | Very Good | ++ | +++ | + | +++ |
| Magnacharge | MSI | NBOHY00010 | Poor | + | +++ | +++++ | + |
| MagnaGraph | MSI | NJOHY00010 | Execellent | − | ++ | +++++ | + |
| Hybond ECL | Amersham | RPN2020D | Excellent | + | +++ | − | +++ |

MSI: Micron separations Inc.

TABLE 3
Comparison of protein levels by protein array and ELISA

| | | Conditioned media | Patient's sera |
|---|---|---|---|
| | | TNFα treated/untreated | No: 4/No: 11 |
| IL-8 | Protein array | 17.0 | 1.1 |
| | ELISA | 24 | 1.3 |
| INFν | Protein array | 1.0 | 1.0 |
| | ELISA | 1.0 | 1.1 |
| MCP-1 | Protein array | 2.5 | 17.0 |
| | ELISA | 2.0 | 22.2 |
| TNFα | Protein array | 8.0 | |
| | ELISA | 8.0 | |

TABLE 4
Variability of human cytokine

| Intra-membrane | | | | Inter-membrane | | | |
|---|---|---|---|---|---|---|---|
| cyto- | Average | | | cyto- | Average | | |
| kine | density | SD | % CV | kine | density | SD | % CV |
| IL-2 | 0.460 | 0.046 | 10.00 | IL-8 | 0.184 | 0.019 | 0.10 |
| IL-6 | 0.706 | 0.066 | 9.36 | MCP-1 | 2.241 | 0.118 | 5.28 |
| INFγ | 0.545 | 0.059 | 11.00 | MCP-2 | 1.412 | 0.115 | 8.18 |
| TNF | 0.730 | 0.045 | 6.26 | Rante | 0.413 | 0.046 | 0.11 |

1.-63. (canceled)

64. A substrate for detecting one or more cytokines in a sample comprising:
a microarray of capture antibodies immobilized on a membrane, wherein the microarray comprises an ordered, two-dimensional arrangement of at least 10 array elements, wherein each array element comprises at least one capture antibody specific for a certain type of cytokine, and wherein at least two array elements are specific for different types of cytokines.

65. The substrate of claim 64, wherein the membrane is made of a material selected from the following: polyvinylidene fluoride, nitrocellulose, and nylon.

66. The substrate of claim 64, wherein the microarray comprises at least 100 array elements.

67. The substrate of claim 64, wherein the cytokines include one or more cytokines selected from the following: chemokines, growth factors, proteases, soluble receptors, and other cytokines.

68. A method for detecting one or more cytokines in a sample comprising:
a) passing a solution containing one or more types of cytokines over a microarray of capture antibodies immobilized on a membrane, wherein the microarray comprises an ordered, two-dimensional arrangement of at least 10 array elements, wherein each array element comprises at least one capture antibody specific for a certain type of cytokine, and wherein at least two array elements are specific for different types of cytokines,
wherein at least one type of cytokine in the solution binds to its respective capture antibody on the membrane;
b) adding a solution of one or more types of labeled detection antibodies to the membrane, wherein at least one type of labeled detection antibody in the solution is capable of binding the bound cytokine; and
c) detecting bound labeled detection antibody, the detection of bound labeled detection antibody in a position on the microarray corresponding to an array element having a capture antibody specific for a certain type of cytokine indicating the presence of that type of cytokine.

69. The method of claim 68, wherein the cytokines include one or more cytokines selected from the following: chemokines, growth factors, proteases, soluble receptors, and other cytokines.

70. The method of claim 68, wherein the membrane is made of a material selected from the following: polyvinylidene fluoride, nitrocellulose, and nylon.

71. The method of claim 68, wherein the microarray comprises at least 100 array elements.

72. A method for detecting one or more specified proteins in a sample comprising:
a) passing a solution containing one or more types of specified protein over a microarray of capture proteins immobilized on a membrane, wherein the microarray comprises an ordered, two-dimensional arrangement of at least 5 array elements, wherein each array element comprises at least one capture protein specific for a certain type of specified protein, and wherein at least two array elements are specific for different types of specified protein,
wherein at least one type of specified protein in the solution binds to its respective capture protein;
b) adding a solution of one or more types of detection protein to the membrane, wherein at least one type of detection protein in the solution is capable of binding the bound specified protein; and
c) detecting bound detection protein, the detection of bound detection protein in a position on the microarray corresponding to an array element having a capture protein specific for a certain type of specified protein indicating the presence of that type of specified protein.

73. The method of claim 72, further comprising washing the membrane to remove unbound specified protein before adding the solution of detection protein.

74. The method of claim 72, further comprising washing the membrane to remove unbound detection protein.

75. The method of claim 72, wherein the capture protein is an antibody.

76. The method of claim 72, wherein the specified protein is an antigen.

77. The method of claim 72, wherein at least one type of specified protein detected is a cytokine.

78. The method of claim 77, wherein the capture protein comprises a cytokine-specific antibody.

79. The method of claim 78, wherein the microarray comprises at least 100 different cytokine-specific capture antibodies able to detect at least 100 different types of cytokines.

80. The method of claim 72, wherein the detection protein comprises an antibody.

81. The method of claim 77, wherein the detection protein comprises a cytokine-specific antibody.

82. The method of claim 77, wherein the capture protein comprises a cytokine-specific antibody specific for a specific epitope of a cytokine and wherein the detection protein comprises a cytokine-specific antibody specific for a different epitope of the cytokine.

83. The method of claim 77, wherein the cytokine includes one or more cytokines selected from the following: chemokines, growth factors, proteases, soluble receptors, and other cytokines

84. The method of claim 72, wherein at least one type of specified protein detected is an angiogenesis factor.

85. The method of claim 72, wherein the membrane is made of a material selected from the following: polyvinylidene fluoride, nitrocellulose, and nylon.

86. The method of claim 72, wherein the specified proteins are unknown proteins from biological samples.

87. The method of claim 72, wherein the microarray has a density of at least 10 array elements/cm²

88. The method of claim 72, wherein the microarray has a density of at least 100 array elements/cm².

89. The method of claim 72, wherein the microarray has a density of at least 1000 array elements/cm².

90. The method of claim 72, wherein the detection protein is biotinylated.

91. The method of claim 88, wherein detecting of bound detection protein comprises adding a solution of a detectable label-conjugated protein capable of binding bound biotinylated detection protein.

92. The method of claim 91 wherein the detectable label-conjugated protein is selected from the following: Cy3-streptavidin, Cy5-streptavidin, streptavidin conjugated with gold particles, radiolabeled streptavidin, Cy3-avidin, Cy5-avidin, avidin conjugated with gold particles, and radiolabeled avidin.

93. The method of claim 72, wherein the detection proteins are horseradish peroxidase-labeled proteins.

94. The method of claim 72, wherein the detection protein is detected by chemiluminescence.

95. The method of claim 94, wherein the detection protein is detected by enhanced chemiluminescence.
